SEQUENCE LISTING (35 sequences)

SEQ ID NO: 1 - DNA, 1344 bp, synthetic construct
  misc_feature 1..1344: note = Synthetic Construct
  source 1..1344: mol_type = other DNA
caggtgcagctgaagcagtctggccctggcctggtgcagccctcccagtccctgtccatcacctgtaccgtgtccggcttctccctgaccaactacggcgtgcactgggtgcgacagtcccccggcaagggcctggaatggctgggagtgatttggagcggcggcaacaccgactacaacacccccttcacctcccggctgtccatcaacaaggacaactccaagtcccaggtgttcttcaagatgaactccctgcagtccaacgacaccgccatctactactgcgccagagccctgacctactatgactacgagttcgcctactggggccagggcaccctggtgacagtgtccgccgcttccaccaagggcccctccgtgttccctctggccccctccagcaagtccacctctggcggcaccgctgccctgggctgcctggtgaaagactacttccccgagcccgtgaccgtgtcctggaactctggcgccctgacctccggcgtgcacaccttccctgccgtgctgcagtcctccggcctgtactccctgtcctccgtggtgaccgtgccctccagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtctgtgctgaccgtgctgcaccaggactggctgaacggcaaagagtacaagtgcaaggtgtccaacaaggccctgcctgcccccatcgaaaagaccatctccaaggccaagggccagccccgcgagccccaggtgtacaccctgccccctagccgggacgagctgaccaagaaccaggtgtccctgacctgtctggtgaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtctctgtcccccggc

SEQ ID NO: 2 - DNA, 642 bp, synthetic construct
  misc_feature 1..642: note = Synthetic Construct
  source 1..642: mol_type = other DNA
gacatcctgctgacccagtcccccgtgatcctgtccgtgtctcctggcgagcgggtgtccttctcctgccgggcctctcagtccatcggcaccaacatccactggtatcagcagcggaccaacggctcccctcggctgctgattaagtacgcctccgagtccatctccggcatcccttcccggttctccggctccggctctggcaccgacttcaccctgtccatcaactccgtggaatccgaggacattgccgactactactgccagcagaacaacaactggcccaccaccttcggcgctggcaccaagctggaactgaagcggaccgtggccgctccctccgtgttcatcttcccaccctccgacgagcagctgaagtccggcaccgcctccgtggtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgt

SEQ ID NO: 3 - DNA, 411 bp, synthetic construct
  misc_feature 1..411: note = Synthetic Construct
  source 1..411: mol_type = other DNA
acaatccctccacacgtgcagaaatccgtgaacaacgacatgatcgtgaccgacaacaatggcgccgtgaagttcccccagctgtgcaagttctgcgacgtgcggttctctacctgcgacaaccagaaatcctgcatgtccaactgctccatcacctccatctgcgagaagccccaggaagtgtgcgtggccgtgtggcggaagaacgacgagaacatcaccctggaaaccgtgtgccacgaccccaagctgccctaccacgacttcatcctggaagatgccgcctcccccaagtgcatcatgaaggaaaagaagaagcccggcgagactttcttcatgtgcagctgctcctccgacgagtgcaacgacaacatcatcttctccgaagagtacaacacctccaaccccgac

SEQ ID NO: 4 - DNA, 45 bp, synthetic construct
  misc_feature 1..45: note = Synthetic Construct
  source 1..45: mol_type = other DNA
ggaggcggaggatctggcggaggtggaagtggcggcggaggctct

SEQ ID NO: 5 - DNA, 1341 bp, synthetic construct
  misc_feature 1..1341: note = Synthetic Construct
  source 1..1341: mol_type = other DNA
caggtgcagctggtggaatctggtggcggagtggtgcagcctggcagatccctgagactgtcttgtgccgcctccggcttcaccttctcctcgtacaccatgcactgggtgcgacaggcccctggcaagggactggaatgggtcaccttcatctcttacgacggcaacaacaagtactacgccgactccgtgaagggccggttcaccatctcccgggacaactccaagaacaccctgtacctgcagatgaactccctgcgggccgaggacaccgccatctactactgtgctagaaccggctggctgggccccttcgattattggggccagggcaccctcgtgaccgtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaacgggtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagatgaactgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccggg

SEQ ID NO: 6 - DNA, 645 bp, synthetic construct
  misc_feature 1..645: note = Synthetic Construct
  source 1..645: mol_type = other DNA
gagatcgtgctgacccagtctcctggcaccctgtctctgagccctggcgagagagctaccctgtcctgcagagcctctcagtccgtgggctcctcttacctggcctggtatcaacaaaaacccggccaagctccccggctgctgatctacggtgccttttctcgcgccaccggcatccccgaccggttctccggatctggctctggcaccgacttcaccctgaccatctcccggctggaacccgaggacttcgccgtgtactactgccagcagtacggctcctccccctggacctttggccagggcaccaaggtggaaatcaaacgtacggtcgcggcgccttccgtgttcatcttcccaccctccgacgagcagctgaagtccggcaccgcctccgtggtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtccagccccgtgaccaagtccttcaaccggggcgagtgt

SEQ ID NO: 7 - DNA, 450 bp, synthetic construct
  misc_feature 1..450: note = Synthetic Construct
  source 1..450: mol_type = other DNA
cctggctggtttctggactcccctgaccggccctggaaccccccaaccttctctcctgccctgctggtggtgacagagggcgacaacgccaccttcacctgttccttcagcaacacctccgagtccttcgtgctgaactggtacagaatgtcccccagcaaccagaccgacaagctggccgccttccccgaggacagatcccagcctggccaggactgccggttcagagtgacccagctgcccaacggccgggacttccacatgtccgtggtgcgagccagacggaacgactccggcacctacctgtgcggcgccatctctctggcccccaaggcccagatcaaagagtccctgcgggccgagctgagagtgaccgagagaagggccgaggtgcccaccgcccaccctagcccatctccaagacctgccggccagttccagaccctggtg

SEQ ID NO: 8 - protein, 448 aa, synthetic construct
  REGION 1..448: note = Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 214 aa, synthetic construct
  REGION 1..214: note = Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 15 aa, synthetic construct
  REGION 1..15: note = Synthetic Construct
GGGGSGGGGSGGGGS

SEQ ID NO: 11 - protein, 150 aa, synthetic construct [sequence omitted]

SEQ ID NO: 12 - protein, 137 aa, synthetic construct
  REGION 1..137: note = Synthetic Construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 13 - protein, 447 aa, synthetic construct
  REGION 1..447: note = Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 215 aa, synthetic construct
  REGION 1..215: note = Synthetic Construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 613 aa, synthetic construct
  REGION 1..613: note = Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 16 - protein, 379 aa, synthetic construct
  REGION 1..379: note = Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 17 - protein, 613 aa, synthetic construct
  REGION 1..613: note = Synthetic Construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 379 aa, synthetic construct
  REGION 1..379: note = Synthetic Construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 28 bp, synthetic construct
  misc_feature 1..28: note = Synthetic Construct
  source 1..28: mol_type = other DNA
agatatcgccaccatgatgtccttcgtg

SEQ ID NO: 20 - DNA, 35 bp, synthetic construct
  misc_feature 1..35: note = Synthetic Construct
  source 1..35: mol_type = other DNA
ggcggcggaggctctcaggtgcagctgaagcagtc

SEQ ID NO: 21 - DNA, 25 bp, synthetic construct
  misc_feature 1..25: note = Synthetic Construct
  source 1..25: mol_type = other DNA
agtatactcagccgggggacagaga

SEQ ID NO: 22 - DNA, 34 bp, synthetic construct
  misc_feature 1..34: note = Synthetic Construct
  source 1..34: mol_type = other DNA
ttcagctgcacctgagagcctccgccgccacttc

SEQ ID NO: 23 - DNA, 35 bp, synthetic construct
  misc_feature 1..35: note = Synthetic Construct
  source 1..35: mol_type = other DNA
attaattaatcaacactcgccccggttgaaggact

SEQ ID NO: 24 - DNA, 35 bp, synthetic construct
  misc_feature 1..35: note = Synthetic Construct
  source 1..35: mol_type = other DNA
ctctgtcccccggcggcggcggaggatctggcgga

SEQ ID NO: 25 - DNA, 35 bp, synthetic construct
  misc_feature 1..35: note = Synthetic Construct
  source 1..35: mol_type = other DNA
gatcctccgccgccgccgggggacagagacaggga

SEQ ID NO: 26 - DNA, 26 bp, synthetic construct
  misc_feature 1..26: note = Synthetic Construct
  source 1..26: mol_type = other DNA
agtatactcacaccagggtctggaac

SEQ ID NO: 27 - protein, 600 aa, synthetic construct
  REGION 1..600: note = Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 28 - protein, 366 aa, synthetic construct
  REGION 1..366: note = Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 29 - protein, 600 aa, synthetic construct
  REGION 1..600: note = Synthetic Construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 366 aa, synthetic construct
  REGION 1..366: note = Synthetic Construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 600 aa, synthetic construct; identical to SEQ ID NO: 27
  REGION 1..600: note = Synthetic Construct

SEQ ID NO: 32 - protein, 612 aa, synthetic construct
  REGION 1..612: note = Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 33 - protein, 380 aa, synthetic construct
  REGION 1..380: note = Synthetic Construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 34 - protein, 612 aa, synthetic construct
  REGION 1..612: note = Synthetic Construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 380 aa, synthetic construct
  REGION 1..380: note = Synthetic Construct
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSGGGGSGGGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0368743 A1
(43) Pub. Date: Dec. 4, 2025

(54) FUSION IMMUNOMODULATORY PROTEINS AND METHODS FOR MAKING SAME

(71) Applicant: Biocon Ltd., Bangalore (IN)

(72) Inventors: Nagaraj GOVINDAPPA, Karnataka (IN); Maria Melina SOARES, Karnataka (IN); Kedarnath SASTRY, Karnataka (IN)

(21) Appl. No.: 19/236,781

(22) Filed: Jun. 12, 2025

Related U.S. Application Data
(63) Continuation of application No. 17/010,178, filed on Sep. 2, 2020, now Pat. No. 12,351,636.
Application No. 17/010,178 is a continuation of application No. 15/968,122, filed on May 1, 2018, now Pat. No. 10,766,963.
Application No. 15/968,122 is a division of application No. 14/771,308, filed on Aug. 28, 2015, now Pat. No. 9,988,456.
(60) Provisional application No. 61/777,016, filed on Mar. 12, 2013.

(51) Int. Cl. C07K 16/28; A61K 38/00; C07K 14/705; C07K 14/71; C12N 15/62

(52) U.S. Cl. CPC C07K 16/2863; C07K 14/705; C07K 14/71; C07K 16/2818; C12N 15/62; A61K 38/00; C07K 2317/40; C07K 2317/526; C07K 2317/90; C07K 2319/00

(57) ABSTRACT

The present invention relates generally to the field of generating recombinant chimeric fusion proteins to be used in the cancer therapy, and more specifically, to fusion molecules of Anti-EGFR1-TGFβRII, Anti-EGFR1-PD1 and Anti-CTLA4-PD1 and methods of generating same, wherein the methods reduce production costs and increase homogeneity of the recombinant chimeric fusion proteins.

### CROSS REFERENCE TO RELATED APPLICATIONS

This application is a continuation application and claims priority to copending U.S. patent application Ser. No. 15/968,122 filed on May 1, 2018 and now U.S. Pat. No. 10,766,963, which in turn is a divisional and claims priority to copending U.S. patent application Ser. No. 14/771,308 filed on Aug. 28, 2015 and now U.S. Pat. No. 9,988,456 which was filed under the provisions of 35 U.S.C. § 371 and claims the priority of International Patent Application No. PCT/US2014/022404 filed on Mar. 10, 2014 which in turn claims priority to U.S. Provisional Patent Application No. 61/777,016 filed on Mar. 12, 2013 the contents of which are hereby incorporated by reference herein for all purposes.

### BACKGROUND OF THE INVENTION

### Technical Field

The present invention relates generally to the field of generating recombinant chimeric fusion proteins to be used in the cancer therapy, and more specifically, to fusion molecules of Anti-EGFR1-TGFβRII, Anti-EGFR1-PD1 and Anti-CTLA4-PD1 and methods of generating same, wherein the methods reduce production costs and increase homogeneity of the recombinant chimeric fusion proteins.

### Related Art

In spite of numerous advances in medical research, cancer remains the second leading cause of death in the United States. Traditional modes of clinical care, such as surgical resection, radiotherapy and chemotherapy, have a significant failure rate, especially for solid tumors. Failure occurs either because the initial tumor is unresponsive, or because of recurrence due to regrowth at the original site or metastasis. Cancer remains a central focus for medical research and development.

Immunotherapy of cancer has been explored for over a century, but it is only in the last decade that various antibody-based products have been introduced into the management of patients with diverse forms of cancer. At present, this is one of the most active areas of clinical research, with numerous antibody therapeutic products already approved in oncology.

Using specific antibodies as therapeutic agents offers advantages over the non-targeted therapies such as systemic chemotherapy via oral or intravenous administration of drugs or radiation therapy. There are two types of antibody-based therapies. The more common type is to identify a tumor antigen (i.e., a protein expressed on tumors and cancer cells and not in normal tissues) and develop an antibody, preferably a monoclonal antibody (mAb) directed to the tumor antigen. One can then conjugate any therapeutic agent, such as a chemotherapeutic agent, a radionuclide, modified toxin, etc., to this antibody to achieve targeted therapy by the therapeutic agent to the tumor. The other kind of antibody based therapy is by providing an antibody which in itself has therapeutic properties against the tumor/cancer cells it targets. The added advantage of this second form of antibody-based therapy is that one may additionally conjugate another therapeutic agent to the therapeutic antibody to achieve a more effective treatment. The major advantage with any antibody-directed therapy and of therapy using monoclonal antibodies (mAbs) in particular, is the ability to deliver increased doses of a therapeutic agent to a tumor, with greater sparing of normal tissue from the side effects of the therapeutic agent.

Despite the identification of several antibodies for cancer therapies, there is still a need to identify new and more effective therapeutics to overcome immune tolerance and activate T cell responses. Further, even though molecular engineering has improved the prospects for such antibody-based therapeutics issues still remain regarding continuity in the generated recombinant products.

### SUMMARY OF THE INVENTION

The present invention provides for a novel and consistent synthesis method for generating homogeneous recombinant fusion immunomodulatory molecules, and more specifically, recombinant chimeric polypeptides including targeting antibodies linked to immunomodulatory proteins.

To mediate an immune response against cancer, T cell activation and co-stimulation are both important. Co-stimulation of T cells is mainly mediated through engaging of CD28 with its ligands of B7 family on antigen presenting cells (APCs). However, after activation, T cells express a molecule called Cytotoxic T-Lymphocyte Antigen 4 (CTLA-4), which binds to B7 ligands with much more affinity than CD28 and such binding down-modulates T cell activity. Thus, including an antibody that binds to the CTLA-4 receptor would block interaction with ligands of the B7 family and enhance anti-tumor response.

Programmed Death Ligand-1 (PDL1), one of the B7 ligands discussed above, obstructs anti-tumor immunity by (i) tolerizing tumor-reactive T cells by binding to its receptor PD1 (CD279) on T cells; (ii) rendering tumor cells resistant to CD8+ T cell and FasL-mediated lysis by PD-1 signaling through tumor cell-expressed PDL1; and (iii) promoting the development and maintenance of induced T regulatory cells. Therefore, PDL1 is a major obstacle to natural anti-tumor immunity and to cancer immunotherapies requiring activation of host T cell-mediated anti-tumor immunity. This concept is supported by studies demonstrating that antibody blocking of PDL1-PD1 interactions improves T cell activation and reduces tumor progression. Although antibodies to PDL1 or PD1 have shown therapeutic efficacy in a subset of cancer patients, the majority of patients do not benefit from antibody treatment. Thus, there is needed a mechanism for regulating PD-L1 function that will lead to a new universally applicable treatment for minimizing PD-L1-mediated immune suppression in cancer patients and that is more effective than currently available mAbs to PD-1 or PD-L1.

A characteristic of many epithelial cancers, such as, cancers of the colon, head and neck, breast, ovary, non-small cell lung (NSCL), and pancreas, is abnormally high levels of epidermal growth factor receptor (EGFR) on the surface of cancer cells. The family of epidermal growth factor receptors (EGFR; HER1, HER2 neu, HER3, and HER4) includes cell membrane receptors with intrinsic tyrosine kinase activity that trigger a cascade of biophysiological signaling reactions in response to the binding of different ligands. These receptors play a key role in the behavior of malignant cells in a variety of human tumors, inducing increased proliferation, decreasing apoptosis, and enhancing tumor cell motility and angiogenesis. Thus, the present invention includes antibodies targeting EGFR family members.

The present invention further provides methods of reducing growth of cancer cells by counteracting immune tolerance of cancer cells, wherein T cells remain active and inhibit the recruitment of T-regulatory that are known to suppress the immune system's response to the tumor. Thus, the chimeric polypeptides generated by the polynucleotides sequences of the present invention are useful for treating cancer because of the expressed fusion or chimeric polypeptides.

In one aspect, the present invention provides for chimeric polypeptides containing at least one targeting moiety to target a cancer cell and at least one immunomodulating moiety that counteracts immune tolerance of cancer cell, wherein the targeting moiety and the immunomodulating moiety are linked by an amino acid spacer of sufficient length of amino acid residues so that both moieties can successfully bond to their individual target. In the alternative, the targeting moiety and the immunomodulating moiety that counteract immune tolerance of cancer cell may be bound directly to each other. The chimeric/fusion polypeptides of the invention are useful for binding to a cancer cell receptor and reducing the ability of cancer cells to avoid an immune response.

Preferably the targeting moiety is an antibody having binding affinity for CTLA-4 or EGFR1, wherein the antibody is transcribed from a polynucleotide sequence lacking nucleotides for expression of the C-terminal lysine of the heavy chain of the expressed antibody. It has been discovered that by removing the C-terminal lysine of the heavy chain of an antibody during transcription that the end product exhibits increased homogeneity, thereby reducing the need and costs for further purification.

It is known that during the process of transcription and translation of an IgG molecule in CHO cells, the lysine (K) at the C-terminal of the heavy chain will be expressed. In the commercial product such expressed lysines have to be removed to increase purity. There is much heterogeneity in the produced product, as shown in FIG. 1. This occurs because the CHO cell has an endogenous enzyme Carboxypeptidase B (CPB) which will cleave the C-terminal lysine as long as the expressed antibody is still available intracellularly. However, this enzyme will not cleave the lysine once the antibody is secreted into the medium. Thus, the cleavage efficiency of this endogenous CPB is based on the availability within the cell. As such, some of the antibodies will be secreted with the lysine and some will not, and such combination will cause significant heterogeneity in the secreted product, that being some antibodies with the C-terminal lysine and some without. As the recombinant product is being used for the therapeutic use, one needs to purify to homogeneity. Thus, the recombinant products of the prior art requires additional purification steps wherein the recombinant product need to be treated with the enzyme CPB first and purified once again using an additional step to remove any lysine and the enzyme CPB from the final product. These additional steps add a significant cost to the manufacturing process.

The present invention avoids the shortcomings of previous methods of synthesizing recombinant anti-CTLA-4 and anti-EGFR1 antibodies by transcribing an expressed protein from a polynucleotide sequence lacking nucleotides for expression of the C-terminal lysine at the heavy chain of the expressed antibody.

The present invention is based on preparing chimeric/fusion proteins by expression of polynucleotides encoding the fusion proteins that counteract or reverse immune tolerance of cancer cells. Cancer cells are able to escape elimination by chemotherapeutic agents or tumor-targeted antibodies via specific immunosuppressive mechanisms in the tumor microenvironment and such ability of cancer cells is recognized as immune tolerance. Such immunosuppressive mechanisms include immunosuppressive cytokines (for example, Transforming growth factor beta (TGF-β)) and regulatory T cells and/or immunosuppressive myeloid dendritic cells (DCs). By counteracting tumor-induced immune tolerance, the present invention provides effective compositions and methods for cancer treatment, optional in combination with another existing cancer treatment. The present invention provides strategies to counteract tumor-induced immune tolerance and enhance the antitumor efficacy of chemotherapy by activating and leveraging T cell-mediated adaptive antitumor against resistant or disseminated cancer cells.

In another aspect, the present invention provides a molecule including at least one targeting moiety fused with at least one immunomodulatory moiety. The targeting moiety specifically binds a target molecule, and the immunomodulatory moiety specifically binds one of the following molecules: (i) Transforming growth factor-beta (TGF-β) and or (ii) Programmed death- 1 ligand 1 (PD-L1).

In a further aspect, the targeting moiety includes an antibody, including both heavy chains and light chains, wherein the antibody specifically binds a component of a tumor cell, tumor antigen, tumor vasculature, tumor microenvironment, or tumor-infiltrating immune cell. Notably the heavy chain and/or light chain may individually be linked to a same type immunomodulatory moiety or a separate and distinct immunomodulatory moiety. Further, a heavy or light chain of an antibody targeting moiety may be linked to an immunomodulatory moiety which in turn can be further linked to a second immunomodulatory moiety wherein there is a linker between the two immunomodulatory moieties.

In a still further aspect, there is provided a chimeric polypeptide that comprised a tumor targeting moiety and an immunomodulatory moiety comprising a molecule that binds transforming growth factor beta (TGF-β), wherein the tumor targeting moiety is an antibody that binds to EGFR1, where in the antibody can be the full antibody, heavy chain or light chain.

The tumor targeting moiety may include monoclonal antibodies that target a cancer cell, including but not limited to cetuximab, trastuzumab, ritubximab, ipilimumab, tremelimumab, muromonab-CD3, abciximab, daclizumab, basiliximab, palivizumab, infliximab. gemtuzumab ozogamicin, alemtuzumab, ibritumomab tiuxetan, adalimumab, omalizumab, tositumomab, I-131 tositumomab, efalizumab, bevacizumab, panitumumab, pertuzumab, natalizumab, etanercept, IGN101 (Aphton), volociximab (Biogen Idec and PDL BioPharm), Anti-CD80 mAb (Biogen Idec), Anti-CD23 mAb (Biogen Idel), CAT-3888 (Cambridge Antibody Technology), CDP-791 (Imclone), eraptuzumab (Immunomedics), MDX-010 (Medarex and BMS), MDX-060 (Medarex), MDX-070 (Medarex), matuzumab (Merck), CP-675,206 (Pfizer), CAL (Roche), SGN-30 (Seattle Genetics), zanolimumab (Serono and Genmab), adecatumumab (Sereno), oregovomab (United Therapeutics), nimotuzumab (YM Bioscience), ABT-874 (Abbott Laboratories), denosumab (Amgen), AM 108 (Amgen), AMG 714 (Amgen), fontolizumab (Biogen Idec and PDL BioPharm), daclizumab (Biogent Idec and PDL BioPharm), golimumab (Centocor and Schering-Plough), CNTO 1275 (Centocor), ocrelizumab (Genetech and Roche), HuMax-CD20 (Genmab), belimumab (HGS and GSK), epratuzumab (Immunomedics), MLN1202 (Millennium Pharmaceuticals), visilizumab (PDL BioPharm), tocilizumab (Roche), ocrerlizumab (Roche), certolizumab pegol (UCB, formerly Celltech), eculizumab (Alexion Pharmaceuticals), pexelizumab (Alexion Pharmaceuticals and Procter & Gamble), abciximab (Centocor), ranibizimumab (Genetech), mepolizumab (GSK), TNX-355 (Tanox), or MYO-029 (Wyeth).

In a preferred embodiment, the tumor targeting moiety is a monoclonal antibody that binds to CTLA-4 or EGFR1 generated by the methods of the present invention, wherein the method comprises the following steps:
a. preparing a codon optimized nucleotide sequence encoding the fusion protein, wherein the codon optimized sequence for the antibody is lacking nucleotides for expression of a lysine at the C-terminal end of the heavy chains of the antibody;
b. cloning the optimized sequence of said fusion protein in a host cell capable of transient or continued expression;
c. growing the host cell in a media under suitable conditions for growing and allowing the host cell to express the fusion protein; and
d. collecting secreted fusion proteins.

In yet another aspect, the immunomodulatory moiety includes a molecule that binds TGF-β and inhibits the function thereof. Specifically the immunomodulatory moiety includes an extracellular ligand-binding domain of Transforming growth factor-beta receptor TGF-βRII, TGF-βRIIb or TGF-βRIII. In another aspect the immunomodulatory moiety includes an extracellular ligand-binding domain (ECD) of TGF-βRII

In a still further aspect, the targeting moiety includes an antibody that specifically binds to HER2/neu, EGFR1, CD20, or cytotoxic T-lymphocyte antigen-4 (CTLA-4) and wherein the immunomodulatory moiety includes an extracellular ligand-binding domain of TGF-βRII.

In yet another aspect, the immunomodulatory moiety includes a molecule that specifically binds to and inhibits the activity of Programmed death-1 ligand 1 (PD-L 1).

In a further aspect, the targeting moiety includes an antibody, antibody fragment, or polypeptide that specifically binds to HER2/neu, EGFR1, CD20, cytotoxic T-lymphocyte antigen-4 (CTLA-4), CD25 (1L-2a receptor; IL-2aR), or CD4 and wherein, the immunomodulatory moiety includes an extracellular ligand-binding domain or ectodomain of Programmed Death-1 (PD-1).

In a still further aspect, the targeting moiety includes an antibody that specifically binds to EGFR1 and CTLA-4, and the immunomodulatory moiety includes a sequence from interacts with transforming growth factor-β (TGF-β).

In one aspect, the present invention provides for optimized genes encoding for a fusion polypeptide comprising at least one targeting moiety and at least one immunomodulatory moiety for treating cancer in a human subject wherein the genes have been optimized to increase expression in a human subject and/or cells.

In another aspect, the present invention provides for a vector comprising optimized genes for treating cancer in a human subject wherein the optimized genes have been modified to increase CG sequences. Preferably, the vector includes nucleotide sequences for encoding at least one targeting moiety, at least one immunomodulatory moiety and a linking moiety, wherein the optimized nucleotide sequences are selected from SEQ ID NOs: 1 to 7, as set forth in FIG. 2.

In yet another aspect, the present invention provides for a method of treating cancer in a subject, the method comprising:
providing at least one recombinant vector comprising nucleotide sequences that encode at least one targeting moiety, at least one immunomodulatory moiety and a linking moiety positioned between the targeting moiety and immunomodulatory moiety, wherein the nucleotide sequences are selected from SEQ ID NOs: 1 to 7; and
administering the recombinant vector to the subject under conditions such that said nucleotide sequences are expressed at a level which produces a therapeutically effective amount of the encoded fusion proteins in the subject.

In yet another aspect, the present invention provides a recombinant host cell transfected with a polynucleotide sequence that encodes a fusion protein peptide of the present invention, wherein the polynucleotide sequences are selected from SEQ ID NOs: 1 to 7.

In a still further aspect, the present invention contemplates a process of preparing a chimeric fusion protein of the present invention comprising:
transfecting a host cell with a polynucleotide sequence that encodes a chimeric fusion protein to produce a transformed host cell, wherein the polynucleotide sequence encodes at least one targeting moiety and at least one immunomodulatory moiety, wherein the polynucleotide sequence comprises a combination of sequences selected from SEQ ID NOs: 1 to 7; and
maintaining the transformed host cell under biological conditions sufficient for expression of the chimeric fusion protein.

In another aspect, the present invention relates to the use of a chimeric fusion protein, wherein the chimeric fusion protein comprises anti-EGFR1 linker PD1 (SEQ ID NOs: 8, 9, 10 and 11); anti-EGFR1-linker-TGFβRII (SEQ ID NOs: 8, 9, 10 and 12); Anti-CTLA-4-linker-PD1 (SEQ ID NOs: 13, 14, 10 and 11), as shown in FIGS. 3, 4 and 5 respectively, in the use of a medicament for the treatment of cancer. Preferably, the fusion protein is expressed in a host cell and such expressed proteins are administered in a therapeutic amount to reduce the effects of cancer in a subject in need thereof.

In a still further aspect, the present invention provides a method of treating a neoplastic disease. The method includes administration to a subject in need thereof one or more fusion proteins of the present invention, in various aspects, the subject is administered one or more fusion protein of the present invention in combination with another anticancer therapy. In one aspect, the anticancer therapy includes a chemotherapeutic molecule, antibody, small molecule kinase inhibitor, hormonal agent or cytotoxic agent. The anticancer therapy may also include ionizing radiation, ultraviolet radiation, cryoablation, thermal ablation, or radiofrequency ablation.

In a preferred embodiment the therapeutically active antibody-peptide fusion proteins is a targeting antibody fused to one or more immunomodulating moiety that counteracts immune tolerance of a cancer cell. In one aspect, the immunomodulating moiety may be linked by an amino acid spacer of sufficient length to allow bi-specific binding of the molecule. The immunomodulating moiety may be bound to either the N-terminus or C-terminus of the heavy chain or the N-terminus or C-terminus of the light chain of the antibody

The method of the present invention provides nucleotide sequences that encode the therapeutically active antibody-peptide fusion proteins and such expression may be conducted in a transient cell line or a stable cell line. The transient expression is accomplished by transfecting or transforming the host cell with vectors carrying the encoded fusion proteins into mammalian host cells

Once the fusion peptides are expressed, they are preferably subjected to purification and in-vitro tests to check its bi-specificity, that being, having the ability to bind to both the target moiety and immunomodulating moiety. Such tests may include in-vitro tests such as ELISA or NK/T-cell binding assays to validate bi-functional target binding or immune cell stimulation.

Notably once the specific fusion peptides demonstrate the desired bi-specificity, the polynucleotide sequences encoding such fusion peptides are selected for sub-cloning into a stable cell line for larger scale expression and purification. Such stable cell lines are previously disclosed, such as a mammalian cell line, including but not limited to HEK293, CHO or NSO.

In another aspect the present invention provides for a method to inhibit and/or reduce binding of PDL1 to PD1 thereby increasing immune response against tumor cells, the method comprising:
a. providing a chimeric polypeptide comprising PD1 and an anti-EGFR1 or anti-CTLA-4 antibody; and
b. contacting a tumor cell with the chimeric polypeptide wherein the chimeric polypeptide binds with at least PDL1 of the tumor cell.

In yet another aspect, the present invention provides for a method of preparing therapeutically active antibody-peptide fusion proteins, the method comprising;
a. preparing a codon optimized sequence of the said fusion protein, wherein the codon optimized sequences for anti-EGFR1 and anti-CTLA-4 antibodies are lacking nucleotides for expression of a lysine at the C-terminal end of the heavy chains of the antibodies;
b. cloning the optimized sequence of said fusion protein in a host cell capable of transient or continued expression;
c. growing the host cell in a media under suitable conditions for growing and allowing the host cell to express the fusion protein; and
d. collecting secreted fusion proteins.

In a still further aspect the present invention provides for a nucleic acid sequence encoding a chimeric fusion protein, wherein the chimeric fusion protein comprises at least one targeting moiety having affinity for a cancer cell and at least one immunomodulatory moiety that counteract immune tolerance of the cancer cell, wherein targeting moiety is an antibody and the nucleic acid sequence of the targeting moiety is lacking nucleotides for expression of a lysine at the C-terminal end of the heavy chains of the antibody. The nucleic acid sequence encoding the heavy chain of the antibody preferably includes SEQ ID NO: 1 or SEQ ID NO:5. The nucleic acid sequence encoding the chimeric fusion proteins preferably comprises a sequence selected from the group consisting of SEQ ID NOs: 1, 2, 4 and 7; SEQ ID NOs: 1, 2, 3 and 4; and SEQ ID NOs: 5, 6, 3 and 4.

In yet another aspect the present invention provides for a method of treating cancer in a subject, the method comprising:
a) preparing a preparing therapeutically active fusion protein, wherein the fusion protein comprises a tumor targeting moiety and at least one immunomodulatory molecule, wherein the tumor targeting moiety is an antibody that binds to CTLA-4 or EGFR1 and wherein the fusion protein is prepared by the following steps:
   i) preparing a codon optimized nucleotide sequence encoding the fusion protein, wherein the codon optimized nucleotide sequence for the antibody is lacking nucleotides for expression of a lysine at the C-terminal end of the heavy chains of the antibody;
   ii) cloning the optimized sequence of said fusion protein in a host cell capable of transient or continued expression;
   iii) growing the host cell in a media under suitable conditions for growing and allowing the host cell to express the fusion protein; and
   iv) collecting secreted fusion proteins;
b) administering a therapeutically active amount of the secreted fusion proteins to the subject.

The fusion protein is selected from the group of amino acid sequences consisting of SEQ ID NOs: 15 and 9; SEQ ID NOs: 8 and 16; SEQ ID NOs: 17 and 9; SEQ ID NOs: 8 and 18; SEQ ID NOs: 27 and 9; SEQ ID NOs: 8 and 28; SEQ ID NOs: 29 and 9; SEQ ID NOs: 8 and 30; SEQ ID NOs: 31 and 28; SEQ ID NOs: 31 and 30; SEQ ID NOs: 29 and 28; SEQ ID NOs: 29 and 30; SEQ ID NOs: 32 and 14; SEQ ID NOs: 13 and 33; SEQ ID NOs: 34 and 14; SEQ ID NOs: 13 and 35; SEQ ID NOs: 32 and 33; SEQ ID NOs: 32 and 35; SEQ ID NOs: 34 and 33 and SEQ ID NOs: 34 and 35.

In another aspect, the present invention provides for a method of treating a neoplastic disease, the method comprising administration to a subject in need thereof one or more fusion proteins encoded by at least one polynucleotide sequence selected from the group consisting of SEQ ID NOs: 1, 2, 4 and 7; SEQ ID NOs: 1, 2, 3 and 4; and SEQ ID NOs: 5, 6, 3 and 4. Notably by using the above defined polynucleotide sequences, the following combination of fusion proteins can be expressed including anti-EGFR1 linker PD1 (SEQ ID NOs: 8, 9, 10 and 11); anti-EGFR1-linker-TGFβRII (SEQ ID NOs: 8, 9, 10 and 12); and Anti-CTLA-4-linker-PD1 (SEQ ID NOs: 13, 14, 10 and 11).

Other features and advantages of the invention will be apparent from the following detailed description, drawings and claims.

### BRIEF DESCRIPTION OF THE FIGURES

FIG. 1 shows the different possibilities of lysine placement on a heavy chain and such heterogeneity causing the need to provide purification.

FIG. 2 shows the optimized codon nucleotide sequences used for expression of the antibody-peptide fusion proteins of the present invention, including Anti-EGFR1 heavy chain (SEQ ID NO: 1); Anti-EGFR1 light chain (SEQ ID NO: 2); PD1 (SEQ ID NO: 7); Linker (SEQ ID NO: 4); Anti-CTLA-4 heavy chain (SEQ ID NO: 5); Anti-CTLA-4 light chain (SEQ ID NO: 6) and TGFβRII (SEQ ID NO: 3).

FIG. 3 shows the amino acid residues for the anti-EGFR1 linker PD1 construct (SEQ ID NOs: 8, 9, 10 and 11).

FIG. 4 shows the amino acid residues for anti-EGFR1-linker-TGFβRII construct (SEQ ID NOs: 8, 9, 10 and 12).

FIG. 5 shows the amino acid residues for the anti-CTLA-4-linker-PD1 (SEQ ID NOs: 13, 14, 10 and 11).

FIG. 6 shows the different possibilities for placement of the PD1 molecule on the anti-EGFR1 antibody for FMab5, FMab6, FMab7 and FMab8.

FIG. 7 shows the amino acid sequences for Anti-EGFR1 HC-PD1+Anti-EGFR1 LC wherein the PD1 molecule is connected to the C terminus of the heavy chain separated by a linker and including SEQ ID NOs: 15 and 9.

FIG. 8 shows the amino acid sequences for Anti-EGFR1 HC +Anti-EGFR1 LC-PD1 wherein the PD1 molecule is connected to the C terminus of the light chain separated by a linker and including SEQ ID NOs: 8 and 16.

FIG. 9 shows the amino acid sequences for Anti-EGFR1 HC+Anti-EGFR1 LC-PD1 wherein the PD1 molecule is connected to the N terminus of the heavy chain separated by a linker and including SEQ ID NOs: 17 and 9.

FIG. 10 shows the amino acid sequences for Anti-EGFR1 HC+PD1-Anti-EGFR1 LC wherein the PD1 molecule is connected to the N terminus of the light chain separated by a linker and including SEQ ID NOs: 8 and 18.

FIG. 11 shows expression constructs developed using the cDNAs as set forth in SEQ ID NOs: 1, 2 and 7.

FIG. 12 shows the different possibilities for placement of the TGFβRII molecule on the anti-EGFR1 antibody, FMab1, FMab2, FMab3, FMab4, FMab9, FMab10, FMab11 and, FMab12.

FIG. 13 shows the amino acid sequences for Anti-EGFR1 HC-TGFβRII+Anti-EGFR1 LC wherein the TGFβRII molecule is connected to the C terminus of the heavy chain separated by a linker and including SEQ ID NOs: 27 and 9.

FIG. 14 shows the amino acid sequences for Anti-EGFR1 HC+Anti-EGFR1 LC-TGFβRII wherein the TGFβRII molecule is connected to the C terminus of the light chain separated by a linker and including SEQ ID NOs: 8 and 28.

FIG. 15 shows the amino acid sequences for TGFβRII-Anti-EGFR1 HC+Anti-EGFR1 LC wherein the TGFβRII molecule is connected to the N terminus of the heavy chain separated by a linker and including SEQ ID NOs: 29 and 9.

FIG. 16 shows the amino acid sequences for Anti-EGFR1 HC+TGFβRII-Anti-EGFR1 LC wherein the TGFβRII molecule is connected to the N terminus of the light chain separated by a linker and including SEQ ID NOs: 8 and 30.

FIG. 17 shows the amino acid sequences for Anti-EGFR1 HC-TGFβRII+Anti-EGFR1 LC-TGFβRII wherein the TGFβRII molecule is connected to the C terminus of the heavy and light chain separated by a linker and including SEQ ID NOs: 31 and 28.

FIG. 18 shows the amino acid sequences for Anti-EGFR1 HC-TGFβRII+TGFβRII-Anti-EGFR1 LC wherein the TGFβRII molecule is connected to the C terminus of the heavy chain and N terminus of the light chain separated by a linker and including SEQ ID NOs: 31 and 30.

FIG. 19 shows the amino acid sequences for TGFβRII-Anti-EGFR1 HC+Anti-EGFR1 LC-TGFβRII wherein the TGFβRII molecule is connected to the N terminus of the heavy chain and C terminus of the light chain separated by a linker and including SEQ ID NOs: 29 and 28.

FIG. 20 shows the amino acid sequences for TGFβRII-Anti-EGFR1 HC+TGFβRII-Anti-EGFR1 LC wherein the TGFβRII molecule is connected to the N terminus of the heavy chain and N terminus of the light chain separated by a linker and including SEQ ID NOs: 29 and 30.

FIG. 21 shows expression constructs developed using the cDNAs as set forth in SEQ ID NOs: 1, 2 and 3.

FIG. 22 shows ProteinA purified samples analyzed on 12% reducing SDS-PAGE.

FIG. 23 shows ProteinA purified samples analyzed on 6% non-reducing SDS-PAGE.

FIG. 24 shows the different possibilities for placement of the PD1 molecule on the anti-CTLA4 antibody.

FIG. 25 shows the amino acid sequences for Anti-CTLA4 HC-PD1+Anti-CTLA4 LC wherein the PD1 molecule is connected to the C terminus of the heavy chain separated by a linker and including SEQ ID NOs: 32 and 14.

FIG. 26 shows the amino acid sequences for Anti-CTLA4 HC+Anti-CTLA4 LC-PD1 wherein the PD1 molecule is connected to the C terminus of the light chain separated by a linker and including SEQ ID NOs: 13 and 33.

FIG. 27 shows the amino acid sequences for PD1-Anti-CTLA4 HC+Anti-CTLA4 LC wherein the PD1 molecule is connected to the N terminus of the heavy chain separated by a linker and including SEQ ID NOs: 34 and 14.

FIG. 28 shows the amino acid sequences for Anti-CTLA4 HC+PD1-Anti-CTLA4 LC wherein the PD1 molecule is connected to the N terminus of the light chain separated by a linker and including SEQ ID NOs: 13 and 35.

FIG. 29 shows the amino acid sequences for Anti-CTLA4 HC-PD1+Anti-CTLA4 LC-PD1 wherein the PD1 molecule is connected to the C terminus of the heavy chain and light chain separated by a linker and including SEQ ID NOs: 32 and 33.

FIG. 30 shows the amino acid sequences for Anti-CTLA4 HC-PD1+PD1-Anti-CTLA4 LC wherein the PD1 molecule is connected to the C terminus of the heavy chain separated by a linker and N terminus of the light chain including SEQ ID NOs: 32 and 35.

FIG. 31 shows the amino acid sequences for PD1-Anti-CTLA4 HC+Anti-CTLA4 LC-PD1 wherein the PD1 molecule is connected to the N terminus of the heavy chain separated by a linker and C terminus of the light chain including SEQ ID NOs: 34 and 33.

FIG. 32 shows the amino acid sequences for PD1-Anti-CTLA4 HC+PD1-Anti-CTLA4 LC wherein the PD1 molecule is connected to the N terminus of the heavy chain separated by a linker and N terminus of the light chain including SEQ ID NOs: 34 and 35.

FIG. 33 shows expression constructs developed using the cDNAs as set forth in SEQ ID NOs: 7, 5 and 6.

FIG. 34 shows EGFR1 target binding ELISA. The Anti-EGFR1 HC-TGFβRII+Anti-EGFR1 LC fusion Mab binds to its immobilized target EGFR1.

FIG. 35 shows TGFβ target binding ELISA. The Anti-EGFR1 HC-TGFβRII+Anti-EGFR1 LC fusion Mab binds to its target TGFβ.

FIG. 36 shows Bifunctional ELISA. The anti-EGFR1 HC-TGFβRII+Anti-EGFR1 LC fusion Mab binds to both its target EGFR1 and TGFβ at the same time.

FIG. 37 shows flow cytometric analysis of the binding of the anti-EGFR1 HC-TGFβRII+Anti-EGFR1 LC fusion Mab to EGFR1-expressing A431 cells.

FIG. 38 shows ADCC against EGFR1-expressing A-431 cells. Anti-EGFR1 HC-TGFβRII+Anti-EGFR1 LC fusion Mab mediates ADCC against EGFR1-expressing A-431 cells and the effect is dose dependent.

FIG. 39 shows Inhibition of proliferation assay. Anti-EGFR1 HC-TGFβRII+Anti-EGFR1 LC fusion Mab inhibits the proliferation of EGFR1-expressing A-431 cells.

FIG. 40 shows EGFR1 target binding ELISA. The Anti-EGFR1 HC+Anti-EGFR1 LC-TGFβRII fusion Mab binds to its immobilized target EGFR1.

FIG. 41 shows TGFP target binding ELISA. The anti-EGFR1 HC+Anti-EGFR1 LC-TGFβRII fusion Mab binds to its target TGFβ.

FIG. 42 shows Bifunctional ELISA. The anti-EGFR1 HC+Anti-EGFR1 LC-TGFβRII fusion Mab binds to both its target EGFR1 and TGFβ at the same time.

FIG. 43 shows Inhibition of proliferation assay. Anti-EGFR1 HC+Anti-EGFR1 LC-TGFβRII fusion Mab inhibits the proliferation of EGFR1-expressing A-431 cells.

FIG. 44 shows EGFR1 target binding ELISA. The TGFβRII-Anti-EGFR1 HC+Anti-EGFR1 LC fusion Mab binds to its immobilized target EGFR1.

FIG. 45 shows TGFβ target binding ELISA. The TGFβRII-anti-EGFR1 HC+anti-EGFR1 LC fusion Mab binds to its target TGFβ.

FIG. 46 shows Bifunctional ELISA. The TGFβRII-Anti-EGFR1 HC+Anti-EGFR1 LC fusion Mab binds to both its target EGFR1 and TGFβ at the same time.

FIG. 47 shows Inhibition of proliferation assay. TGFβRII-Anti-EGFR1 HC+Anti-EGFR1 LC fusion Mab inhibits the proliferation of EGFR1-expressing A-431 cells.

FIG. 48 shows EGFR1 target binding ELISA. The Anti-EGFR1 HC+TGFβRII-Anti-EGFR1 LC fusion Mab binds to its immobilized target EGFR1.

FIG. 49 shows TGFβ target binding ELISA. The Anti-EGFR1 HC+TGFβRII-Anti-EGFR1 LC fusion Mab binds to its target TGFβ.

FIG. 50 shows Bifunctional ELISA. The Anti-EGFR1 HC+TGFβRII-Anti-EGFR1 LC fusion Mab binds to both its target EGFR1 and TGFβ at the same time.

FIG. 51 shows flow cytometric analysis of the binding of the Anti-EGFR1 HC+TGFβRII-Anti-EGFR1 LC fusion Mab to EGFR1-expressing A431 cells.

FIG. 52 shows EGFR1 target binding ELISA. The Anti-EGFR1 HC-TGFβRII+TGFβRII-Anti-EGFR1 LC fusion Mab binds to its immobilized target EGFR1.

FIG. 53 shows TGFβ target binding ELISA. The Anti-EGFR1 HC-TGFβRII+TGFβRII-Anti-EGFR1 LC fusion Mab binds to its target TGFβ.

FIG. 54 shows Bifunctional ELISA. The Anti-EGFR1 HC-TGFβRII+TGFβRII-Anti-EGFR1 LC fusion Mab binds to both its target EGFR1 and TGFβ at the same time.

FIG. 55 shows EGFR1 target binding ELISA. The TGFβRII-Anti-EGFR1 HC+TGFβRII-Anti-EGFR1 LC fusion Mab binds to its immobilized target EGFR1.

FIG. 56 shows TGFβ target binding ELISA. TGFβRII-Anti-EGFR1 HC+TGFβRII-Anti-EGFR1 LC fusion Mab binds to its target TGFβ.

FIG. 57 shows Bifunctional ELISA. The TGFβRII-Anti-EGFR1 HC+TGFβRII-Anti-EGFR1 LC fusion Mab binds to both its target EGFR1 and TGFβ at the same time.

FIG. 58 shows Bifunctional ELISA. The Anti-CTLA4 HC-PD1+Anti-CTLA4 LC fusion Mab binds to both its target CTLA4 and PDL1 at the same time.

FIG. 59 shows Bifunctional ELISA. The Anti-CTLA4 HC+Anti-CTLA4 LC-PD1 fusion Mab binds to both its target CTLA4 and PDL1 at the same time.

FIG. 60 shows Bifunctional ELISA. The PD1-Anti-CTLA4 HC+Anti-CTLA4 LC fusion Mab binds to both its target CTLA4 and PDL1 at the same time.

FIG. 61 shows Bifunctional ELISA. The Anti-CTLA4 HC-PD1+PD1-Anti-CTLA4 LC-PD1, Anti-CTLA4 HC-PD1+Anti-CTLA4 LC-PD1 and Anti-CTLA4 HC-PD1+PD1-Anti-CTLA4 LC fusion Mabs binds to both their target CTLA4 and PDL1 at the same time.

FIG. 62 shows Bifunctional ELISA. The PD1-Anti-CTLA4 HC+Anti-CTLA4 LC-PD1 and PD1-Anti-CTLA4 HC+PD1-Anti-CTLA4 LC fusion Mabs binds to both their target CTLA4 and PDL 1 at the same time.

FIG. 63 shows Bifunctional ELISA. The Anti-EGFR1 HC-PD1+Anti-EGFR1 LC fusion Mab binds to both its target EGFR and PDL1 at the same time.

FIG. 64 shows Bifunctional ELISA. The Anti-EGFR1 HC+Anti-EGFR1 LC-PD1 fusion Mab binds to both its target EGFR and PDL1 at the same time.

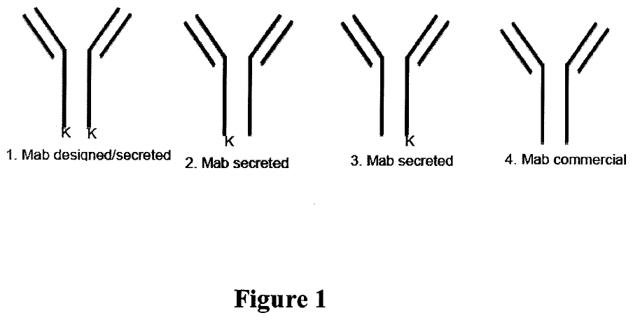

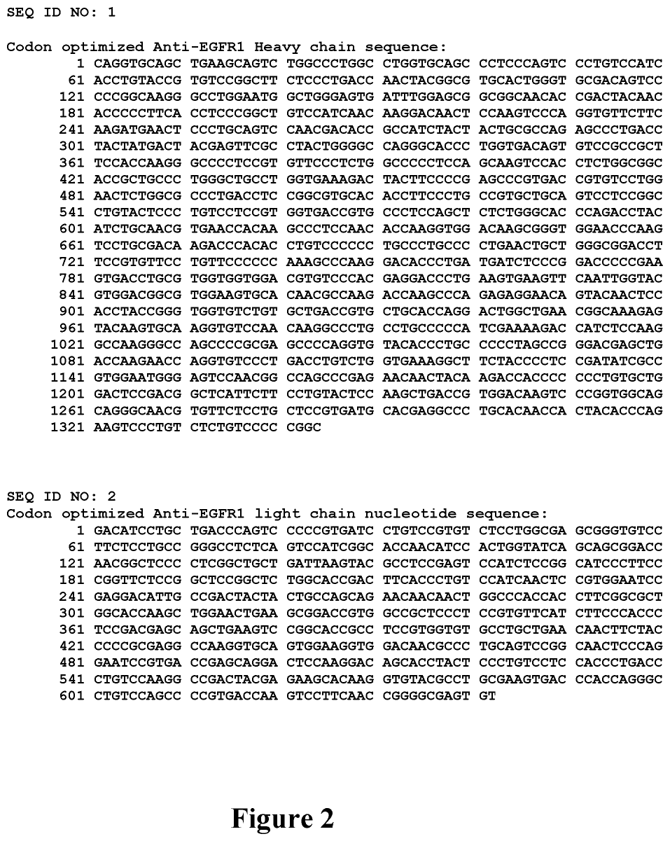

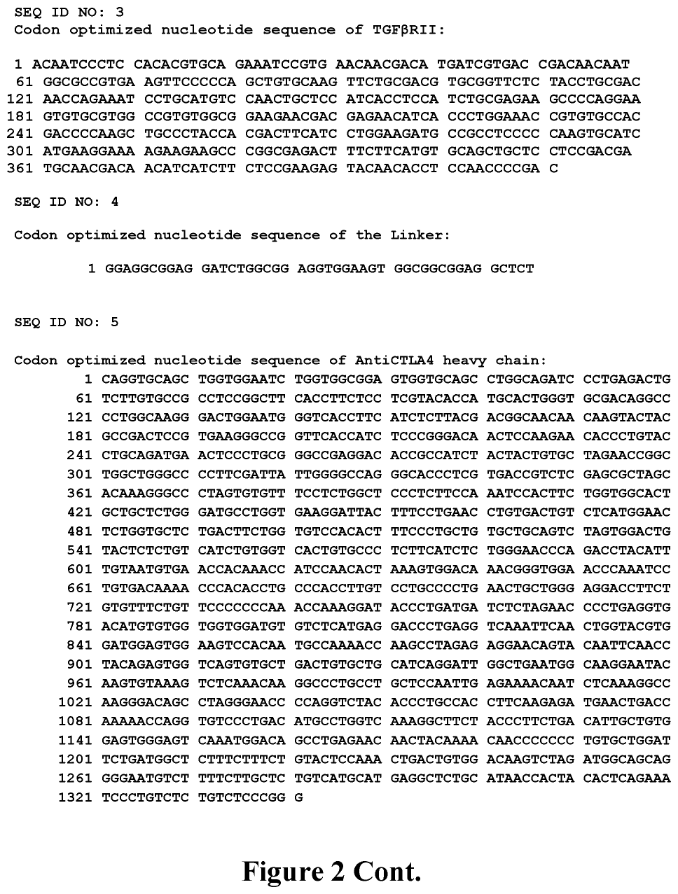

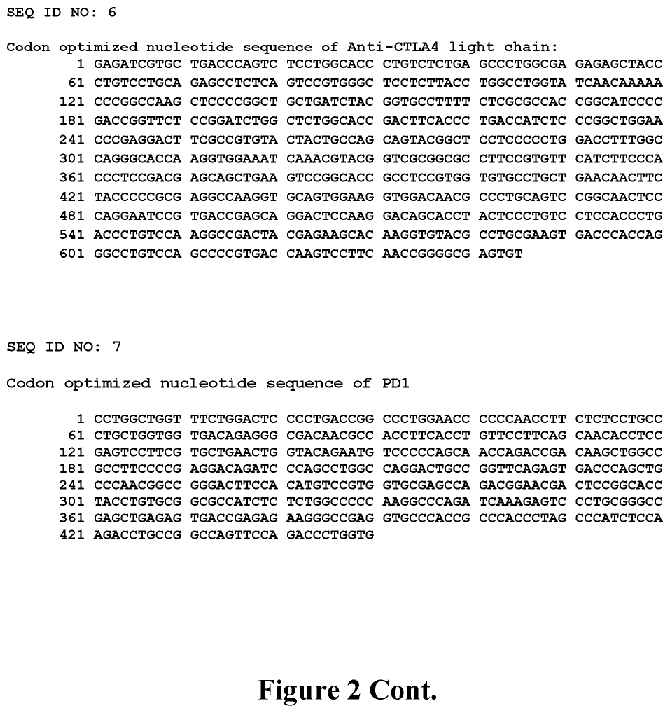

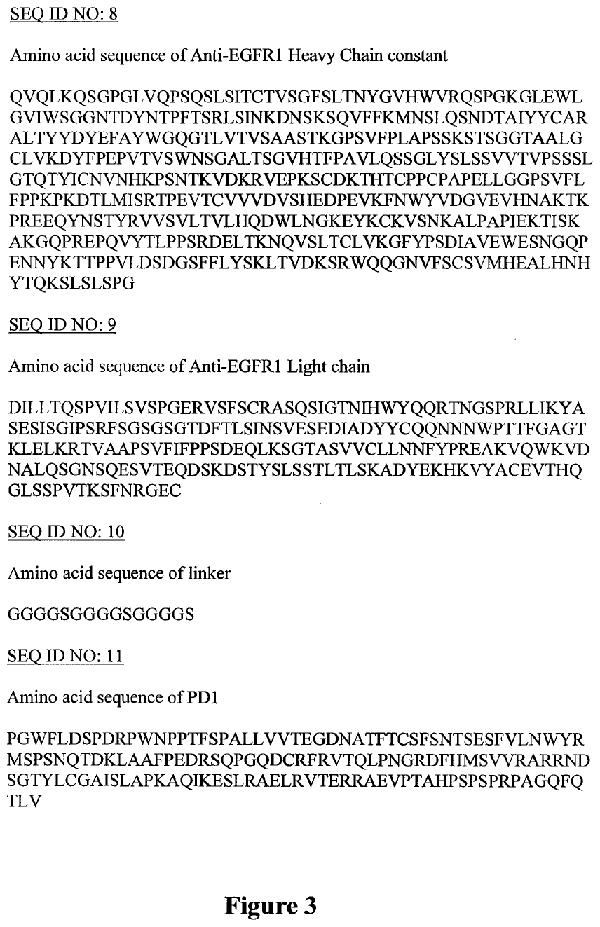

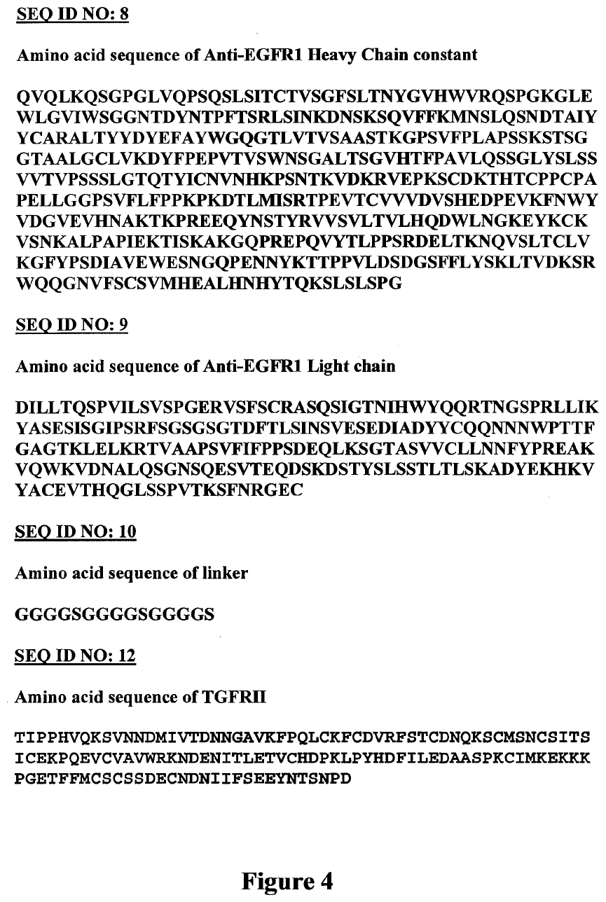

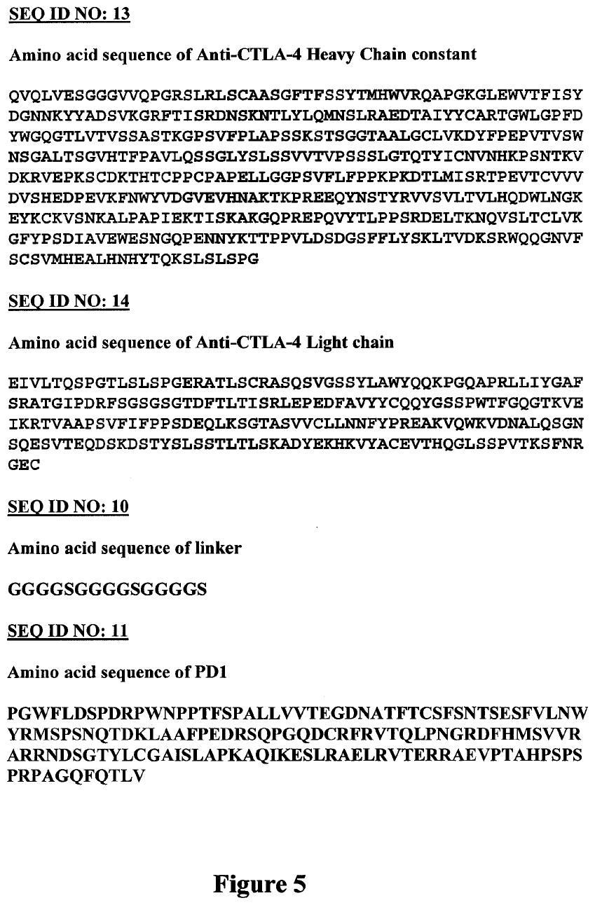

### DETAILED DESCRIPTION OF THE INVENTION

In order to facilitate review of the various embodiments of the invention and provide an understanding of the various elements and constituents used in making and using the present invention, the following terms used in the invention description have the following meanings.

As used herein, the terms “polypeptide,” “protein” and “peptide” are used interchangeably to denote a sequence polymer of at least two amino acids covalently linked by an amide bond, regardless of length or post-translational modification (e.g., glycosylation, phosphorylation, lipidation, myristilation, ubiquitination, etc.). D- and L-amino acids, and mixtures of D- and L-amino acids are also included.

Chimeric polypeptide refers to an amino acid sequence having two or more parts which generally are not found together in an amino acid sequence in nature.

The term “spacer/linker” as used herein refers to a molecule that connects two monomeric protein units to form a chimeric molecule and still provides for binding of the parts to the desired receptors. Particular examples of spacer/linkers may include an amino acid spacer, wherein thee amino acid sequence can essentially be any length, for example, as few as 5 or as many as 200 or more preferably from about 5 to 30 amino acid residues.

The term “therapeutic,” as used herein, means a treatment administered to a subject who exhibits signs of pathology for the purpose of diminishing or eliminating those signs.

The term “therapeutically effective amount,” as used herein means an amount of the chimeric protein that is sufficient to provide a beneficial effect to the subject to which the chimeric protein is administered.

Another example of a modification is the addition of a heterologous domain that imparts a distinct functionality upon the chimeric polypeptide. A heterologous domain can be any small organic or inorganic molecule or macromolecule, so long as it imparts an additional function. Particular examples of heterologous domains that impart a distinct function include an amino acid sequence that imparts targeting (e.g., receptor ligand, antibody, etc.), immunopotentiating function (e.g., immunoglobulin, an adjuvant), enable purification, isolation or detection (e.g., myc, T7 tag, polyhistidine, avidin, biotin, lectins, etc.).

As exemplified herein, polypeptide sequences may include substitutions, variations, or derivitizations of the amino acid sequence of one or both of the polypeptide sequences that comprise the chimeric polypeptide, so long as the modified chimeric polypeptide has substantially the same activity or function as the unmodified chimeric polypeptide.

As used herein, the term “substantially the same activity or function,” when used in reference to a chimeric polypeptide so modified, means that the polypeptide retains most, all or more of the activity associated with the unmodified polypeptide, as described herein or known in the art.

Modified chimeric polypeptides that are “active” or “functional” included herein can be identified through a routine functional assay. For example, by using antibody binding assays or co-receptor binding assays one can readily determine whether the modified chimeric polypeptide has activity. As the modified chimeric polypeptides will retain activity or function associated with unmodified chimeric polypeptide, modified chimeric polypeptides will generally have an amino acid sequence “substantially identical” or “substantially homologous” with the amino acid sequence of the unmodified polypeptide.

As used herein, the term “substantially identical” or “substantially homologous,” when used in reference to a polypeptide sequence, means that a sequence of the polypeptide is at least 50% identical to a reference sequence. Modified polypeptides and substantially identical polypeptides will typically have at least 70%, alternatively 85%, more likely 90%, and most likely 95% homology to a reference polypeptide.

As set forth herein, substantially identical or homologous polypeptides include additions, truncations, internal deletions or insertions, conservative and non-conservative substitutions, or other modifications located at positions of the amino acid sequence which do not destroy the function of the chimeric polypeptide (as determined by functional assays, e.g., as described herein). A particular example of a substitution is where one or more amino acids are replaced by another, chemically or biologically similar residue. As used herein, the term “conservative substitution” refers to a substitution of one residue with a chemically or biologically similar residue. Examples of conservative substitutions include the replacement of a hydrophobic residue, such as isoleucine, valine, leucine, or methionine for another, the replacement of a polar residue for another, such as the substitution of arginine for lysine, glutamic for aspartic acids, or glutamine for asparagine, and the like. Those of skill in the art will recognize the numerous amino acids that can be modified or substituted with other chemically similar residues without substantially altering activity.

Modified polypeptides further include “chemical derivatives,” in which one or more of the amino acids therein have a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxyl groups that form 0-acyl or 0-alkyl derivatives, as well as naturally occurring amino acid derivatives, for example, 4-hydroxyproline, for proline, 5-hydroxylysine for lysine, homoserine for serine, ornithine for lysine, and so forth. Also included are D-amino acids and amino acid derivatives that can alter covalent bonding, for example, the disulfide linkage that forms between two cysteine residues that produces a cyclized polypeptide.

As used herein, the terms “isolated” or “substantially pure,” when used as a modifier of invention chimeric polypeptides, sequence fragments thereof, and polynucleotides, means that they are produced by human intervention and are separated from their native in vivo-cellular environment. Generally, polypeptides and polynucleotides so separated are substantially free of other proteins, nucleic acids, lipids, carbohydrates or other materials with which they are naturally associated.

Polypeptides of the present invention may be prepared by standard techniques well known to those skilled in the art. Such techniques include, but are not limited to, isolation and purification from tissues known to contain that polypeptide, and expression from cloned DNA that encodes such a polypeptide using transformed cells. Chimeric polypeptides can be obtained by expression of a polynucleotide encoding the polypeptide in a host cell, such as a bacteria, yeast or mammalian cell, and purifying the expressed chimeric polypeptide by purification using typical biochemical methods (e.g., immunoaffinity purification, gel purification, expression screening etc.). Other well-known methods are described in Deutscher et al., 1990. Alternatively, the chimeric polypeptide can be chemically synthesized. Purity can be measured by any appropriate method, e.g., polyacrylamide gel electrophoresis, and subsequent staining of the gel (e.g., silver stain) or by HPLC analysis.

The present invention further provides polynucleotide sequences encoding chimeric polypeptides, fragments thereof, and complementary sequences. As used herein, the terms “nucleic acid,” “polynucleotide,” “oligonucleotide,” and “primer” are used interchangeably to refer to deoxyribonucleic acid (DNA) or ribonucleic (RNA), either double- or single-stranded, linear or circular. RNA can be unspliced or spliced mRNA, rRNA, tRNA, or antisense RNAi. DNA can be complementary DNA (cDNA), genomic DNA, or an antisense. Specifically included are nucleotide analogues and derivatives, such as those that are resistant to nuclease degradation, which can function to encode an invention chimeric polypeptide. Nuclease resistant oligonucleotides and polynucleotides are particularly useful for the present nucleic acid vaccines described herein.

An “isolated” or “substantially pure” polynucleotide means that the nucleic acid is not immediately contiguous with the coding sequences with either the 5′ end or the 3′ end with which it is immediately contiguous in the naturally occurring genome of the organism from which it is derived. The term therefore includes, for example, a recombinant DNA (e.g., a cDNA or a genomic DNA fragment produced by PCR or restriction endonuclease treatment produced during cloning), as well as a recombinant DNA incorporated into a vector, an autonomously replicating plasmid or virus, or a genomic DNA of a prokaryote or eukaryote.

The polynucleotides sequences of the present invention can be obtained using standard techniques known in the art (e.g., molecular cloning, chemical synthesis) and the purity can be determined by polyacrylamide or agarose gel electrophoresis, sequencing analysis, and the like. Polynucleotides also can be isolated using hybridization or computer-based techniques that are well known in the art. Such techniques include, but are not limited to: (1) hybridization of genomic DNA or cDNA libraries with probes to detect homologous nucleotide sequences; (2) antibody screening of polypeptides expressed by DNA sequences (e.g., using an expression library); (3) polymerase chain reaction (PCR) of genomic DNA or cDNA using primers capable of annealing to a nucleic acid sequence of interest; (4) computer searches of sequence databases for related sequences; and (5) differential screening of a subtracted nucleic acid library.

The invention also includes substantially homologous polynucleotides. As used herein, the term “homologous,” when used in reference to nucleic acid molecule, refers to similarity between two nucleotide sequences. When a nucleotide position in both of the molecules is occupied by identical nucleotides, then they are homologous at that position. “Substantially homologous” nucleic acid sequences are at least 50% homologous, more likely at least 75% homologous, and most likely 90% or more homologous. As with substantially homologous invention chimeric polypeptides, polynucleotides substantially homologous to invention polynucleotides encoding chimeric polypeptides encode polypeptides that retain most or all of the activity or function associated with the sequence to which it is homologous. For polynucleotides, the length of comparison between sequences will generally be at least 30 nucleotides, alternatively at least 50 nucleotides, more likely at least 75 nucleotides, and most likely 110 nucleotides or more. Algorithms for identifying homologous sequences that account for polynucleotide sequence gaps and mismatched oligonucleotides are known in the art, such as BLAST (see Altschul, 1990).

The polynucleotides of the present invention can, if desired: be naked or be in a carrier suitable for passing through a cell membrane (e.g., polynucleotide-liposome complex or a colloidal dispersion system), contained in a vector (e.g., retrovirus vector, adenoviral vectors, and the like), linked to inert beads or other heterologous domains (e.g., antibodies, ligands, biotin, streptavidin, lectins, and the like), or other appropriate compositions disclosed herein or known in the art. Thus, viral and non-viral means of polynucleotide delivery can be achieved and are contemplated. The polynucleotides of the present invention can also contain additional nucleic acid sequences linked thereto that encode a polypeptide having a distinct functionality, such as the various heterologous domains set forth herein.

The polynucleotides of the present invention can also be modified, for example, to be resistant to nucleases to enhance their stability in a pharmaceutical formulation. The described polynucleotides are useful for encoding chimeric polypeptides of the present invention, especially when such polynucleotides are incorporated into expression systems disclosed herein or known in the art. Accordingly, polynucleotides including an expression vector are also included.

For propagation or expression in cells, polynucleotides described herein can be inserted into a vector. The term “vector” refers to a plasmid, virus, or other vehicle known in the art that can be manipulated by insertion or incorporation of a nucleic acid. Such vectors can be used for genetic manipulation (i.e., “cloning vectors”) or can be used to transcribe or translate the inserted polynucleotide (i.e., “expression vectors”). A vector generally contains at least an origin of replication for propagation in a cell and a promoter. Control elements, including promoters present within an expression vector, are included to facilitate proper transcription and translation (e.g., splicing signal for introns, maintenance of the correct reading frame of the gene to permit in-frame translation of mRNA and stop codons). In vivo or in vitro expression of the polynucleotides described herein can be conferred by a promoter operably linked to the nucleic acid.

“Promoter” refers to a minimal nucleic acid sequence sufficient to direct transcription of the nucleic acid to which the promoter is operably linked (see Bitter 1987). Promoters can constitutively direct transcription, can be tissue-specific, or can render inducible or repressible transcription; such elements are generally located in the 5′ or 3′ regions of the gene so regulated.

As used herein, the term “operably linked” means that a selected polynucleotide (e.g., encoding a chimeric polypeptide) and regulatory sequence(s) are connected in such a way as to permit transcription when the appropriate molecules (e.g., transcriptional activator proteins) are bound to the regulatory sequence(s). Typically, a promoter is located at the 5′ end of the polynucleotide and may be in close proximity of the transcription initiation site to allow the promoter to regulate expression of the polynucleotide.

When cloning in bacterial systems, constitutive promoters, such as T7 and the like, as well as inducible promoters, such as pL of bacteriophage gamma, plac, ptrp, ptac, may be used. When cloning in mammalian cell systems, constitutive promoters, such as SV40, RSV and the like, or inducible promoters derived from the genome of mammalian cells (e.g., the metallothionein promoter) or from mammalian viruses (e.g., the mouse mammary tumor virus long terminal repeat, the adenovirus late promoter), may be used. Promoters produced by recombinant DNA or synthetic techniques may also be used to provide for transcription of the nucleic acid sequences of the invention.

Mammalian expression systems that utilize recombinant viruses or viral elements to direct expression may be engineered. For example, when using adenovirus expression vectors, the nucleic acid sequence may be ligated to an adenovirus transcription/translation control complex, e.g., the late promoter and tripartite leader sequence. Alternatively, the vaccinia virus 7.5K promoter may be used (see Mackett 1982; Mackett 1984; Panicali 1982).

For yeast expression, a number of vectors containing constitutive or inducible promoters may be used (see Ausubel 1988; Grant 1987; Glover 1986; Bitter 1987; and Strathem 1982). The polynucleotides may be inserted into an expression vector for expression in vitro (e.g., using in vitro transcription/translation kits, which are available commercially), or may be inserted into an expression vector that contains a promoter sequence that facilitates expression in either prokaryotes or eukaryotes by transfer of an appropriate nucleic acid into a suitable cell, organ, tissue, or organism in vivo.

As used herein, a “transgene” is any piece of a polynucleotide inserted by artifice into a host cell, and becomes part of the organism that develops from that cell. A transgene can include one or more promoters and any other DNA, such as introns, necessary for expression of the selected DNA, all operably linked to the selected DNA, and may include an enhancer sequence. A transgene may include a polynucleotide that is partly or entirely heterologous (i.e., foreign) to the transgenic organism, or may represent a gene homologous to an endogenous gene of the organism. Transgenes may integrate into the host cell's genome or be maintained as a self-replicating plasmid.

As used herein, a “host cell” is a cell into which a polynucleotide is introduced that can be propagated, transcribed, or encoded polypeptide expressed. The term also includes any progeny of the subject host cell. It is understood that all progeny may not be identical to the parental cell, since there may be mutations that occur during replication. Host cells include but are not limited to bacteria, yeast, insect, and mammalian cells. For example, bacteria transformed with recombinant bacteriophage polynucleotide, plasmid nucleic acid, or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV), or transformed with recombinant plasmid expression vectors (e.g., Ti plasmid), insect cell systems infected with recombinant virus expression vectors (e.g., baculovirus), or animal cell systems infected with recombinant virus expression vectors (e.g., retroviruses, adenovirus, vaccinia virus), or transformed animal cell systems engineered for stable expression.

As used herein, the term “transformation” means a genetic change in a cell following incorporation of a polynucleotide (e.g., a transgene) exogenous to the cell. Thus, a “transformed cell” is a cell into which, or a progeny of which, a polynucleotide has been introduced by means of recombinant techniques. Transformation of a host cell may be carried out by conventional techniques known to those skilled in the art. When the host cell is a eukaryote, methods of DNA transformation include, for example, calcium phosphate, microinjection, electroporation, liposomes, and viral vectors. Eukaryotic cells also can be co-transformed with invention polynucleotide sequences or fragments thereof, and a second DNA molecule encoding a selectable marker, as described herein or otherwise known in the art. Another method is to use a eukaryotic viral vector, such as simian virus 40 (SV40) or bovine papilloma virus, to transiently infect or transform eukaryotic cells, and express the protein (see Gluzman 1982). When the host is prokaryotic (e.g., E. coli), competent cells that are capable of DNA uptake can be prepared from cells harvested after exponential growth phase and subsequently treated by the CaCl₂ method using procedures well-known in the art. Transformation of prokaryotes also can be performed by protoplast fusion of the host cell.

Chimeric polypeptides, polynucleotides, and expression vectors containing same of the present invention can be encapsulated within liposomes using standard techniques and introduced into cells or whole organisms. Cationic liposomes are preferred for delivery of polynucleotides. The use of liposomes for introducing various compositions in vitro or in vivo, including proteins and polynucleotides, is known to those of skill in the art.

Liposomes can be targeted to a cell type or tissue of interest by the addition to the liposome preparation of a ligand, such as a polypeptide, for which a corresponding cellular receptor has been identified. Monoclonal antibodies can also be used for targeting; many such antibodies specific for a wide variety of cell surface proteins are known to those skilled in the art and are available. The selected ligand is covalently conjugated to a lipid anchor in either preformed liposomes or are incorporated during liposome preparation (see Lee 1994 and Lee 1995).

As the chimeric polypeptides or polynucleotides of the present invention will be administered to humans, the present invention also provides pharmaceutical formulations comprising the disclosed chimeric polypeptides or polynucleotides. The compositions administered to a subject will therefore be in a “pharmaceutically acceptable” or “physiologically acceptable” formulation.

As used herein, the terms “pharmaceutically acceptable” and “physiologically acceptable” refer to carriers, diluents, excipients, and the like that can be administered to a subject, preferably without excessive adverse side effects (e.g., nausea, headaches, etc.). Such preparations for administration include sterile aqueous or non-aqueous solutions, suspensions, and emulsions. Examples of non-aqueous solvents are propylene glycol, polyethylene glycol, vegetable oils, such as olive oil, and injectable organic esters, such as ethyl oleate. Aqueous carriers include water, alcoholic/aqueous solutions, emulsions, or suspensions, including saline and buffered media. Vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present, such as, for example, antimicrobial, anti-oxidants, chelating agents, and inert gases and the like. Various pharmaceutical formulations appropriate for administration to a subject known in the art are applicable in the methods of the invention (e.g., Remington's Pharmaceutical Sciences, 18th ed., Mack Publishing Co., Easton, PA (1990); and The Merck Index, 12th ed., Merck Publishing Group, Whitehouse, NJ (1996)).

Controlling the duration of action or controlled delivery of an administered composition can be achieved by incorporating the composition into particles or a polymeric substance, such as polyesters, polyamine acids, hydrogel, polyvinyl pyrrolidone, ethylene-vinylacetate, methylcellulose, carboxymethylcellulose, protamine sulfate or lactide/glycolide copolymers, polylactide/glycolide copolymers, or ethylenevinylacetate copolymers. The rate of release of the composition may be controlled by altering the concentration or composition of such macromolecules. Colloidal dispersion systems include macromolecule complexes, nano-capsules, microspheres, beads, and lipid-based systems, including oil-in-water emulsions, micelles, mixed micelles, and liposomes.

The compositions administered by a method of the present invention can be administered parenterally by injection, by gradual perfusion over time, or by bolus administration or by a microfabricated implantable device. The composition can be administered via inhalation, intravenously, intraperitoneally, intramuscularly, subcutaneously, intracavity (e.g., vaginal or anal), transdermally, topically, or intravascularly. The compositions can be administered in multiple doses. An effective amount can readily be determined by those skilled in the art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. Other features and advantages of the invention will be apparent from the following detailed description, and from the claims. The invention is further described in the following examples, which do not limit the scope of the invention(s) described in the claims.

### EXAMPLES

### 1. Anti-EGFR1-PD1 Fusion Protein Constructs for Cancer Targets

Anti-EGFR (Cetuximab) has been approved for squamous Head and Neck Cancer (locally or regionally advanced in combination with radiotherapy and metastatic after platinum based therapy) and EGFR expressing metastatic colorectal cancer (monotherapy in patients after failure of both oxaliplatin and irinotecan based chemo or in patients intolerant to irinotecan based chemo). Not applicable for colonrectal cancer (CRC) patients having K-RAS mutations.

Across various studies about 55-60% of mCRC patients respond to cetuximab in first line setting, however, this response too is transient (progression free survival (PFS) advantage of 1.5-2 mths) (EPAR). Significant numbers of patients either do not respond to cetuximab or become resistant to therapy. In the recurrent metastatic head and neck cancer, only 35% patients respond to cetuximab with chemo with only 2-3 month overall survival (OS) and (PFS) advantage.

Clearly, a significant unmet need exists to improve efficacy of cetuximab therapy in both these indications. Moreover, EGFR is also expressed in gastric cancer, non-small cell lung cancer (NSCLC) and pancreatic cancers. However, cetuximab has failed to prove any significant benefit in these indications over standard of care. Thus, the present invention provides for improvement by combining cetuximab with an immunomodulatory therapy.

Programmed death-1 (PD-1) is an inhibitory receptor expressed on T cells after activation. It has been shown to down-regulate T-cell activity upon binding its ligand PD-L1 on APCs. Many tumors constitutively express PD-L1 and its' over expression has been associated with impaired tumor immunity, more aggressive disease and decreased survival (see Thompson 2004). Till date PD-L1 expression has been demonstrated to correlate with poor prognosis in patients with renal cell carcinoma (RCC), ovarian cancer and melanoma. Immunohistochemical analysis of freshly isolated tumor samples from patients with ovarian, lung, and breast cancers, renal cell carcinoma, squamous cell carcinoma of the head and neck, esophageal carcinoma, glioblastoma, thymoma, colon carcinoma, pancreatic and melanoma found that the vast majority express B7-H1 (see Flies 2011; Nomi 2007). Several pre-clinical studies have demonstrated increased tumor rejection by blocking PD1-PDL1 interaction. Recently, anti-PD1 and PD-L1 based therapies have demonstrated considerable activity in melanoma and some other solid tumors confirming their application as one of the most promising anti-cancer therapies.

Cetuximab based therapy may be improved upon by combining it with immunomodulation to remove immunosuppressive environment or delay the development of resistance. Moreover, patients who develop resistance to cetuximab due to mutations in the downstream pathways may still benefit from Anti-EGFR1-PD-1 since the fusion protein of the present invention binds to the EGFR receptor and negates the PD-L1 expressed by the tumors, allowing T cells to mount an anti-tumor response. Accordingly, the fusion proteins of the present invention can bind to both EGFR and PD-L1 on the surface of the tumor cells.

The anti-EGFR1-PD1 fusion protein constructs of the present invention may be used in colorectal cancer, squamous head and neck cancer, non-small cell lung cancer, gastric cancer and pancreatic cancer.

### Design and Selection of the Molecules:

The antibody fusion molecules of the present invention have duel therapeutic properties. On one hand the molecule retains the complete activity of the Anti-EGFR1 (Cetuximab) and in parallel, it has the PD-L1 receptor binding activity in the tumor environment. The new molecules of the Anti-EGFR1-PD1 fusion proteins that were developed for the cancer therapies herein are devoid of the amino acid lysine ‘K’ from the C-terminus of heavy chain for the reasons described above. The main objective of the fusion protein design is to keep the anti-EGFR1 molecule intact along with its function unaffected and allows fusion of the PD1 molecule to the various location on the anti-EGFR1 antibody. That being, fusion to the HC C-terminus, LC C-terminus, HC N-terminus, and or LC N-terminus and double fusions on both the chains as shown in FIG. 6.

The following constructs were designed.

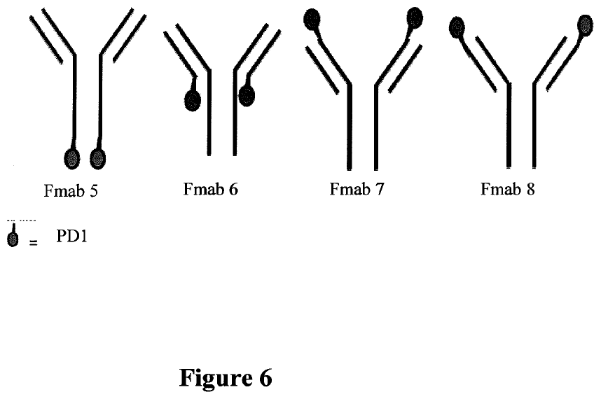

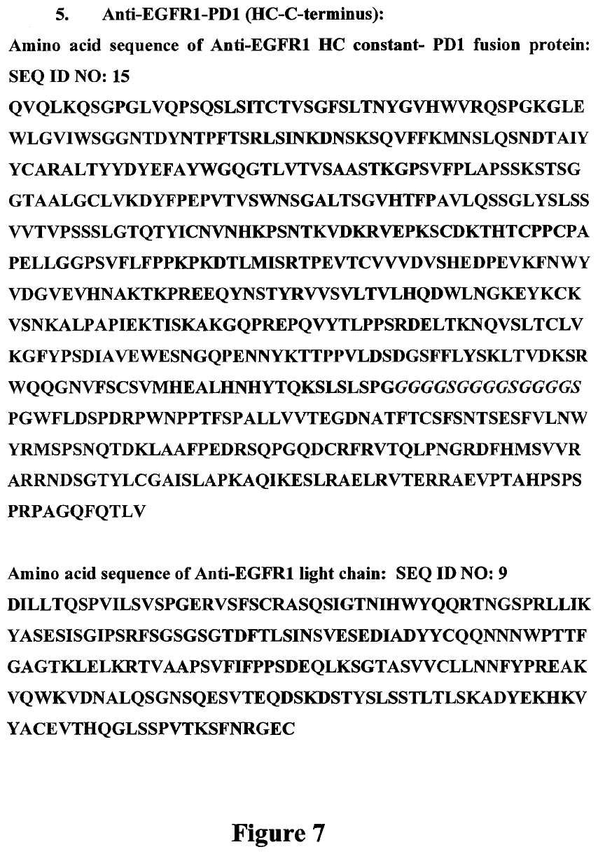

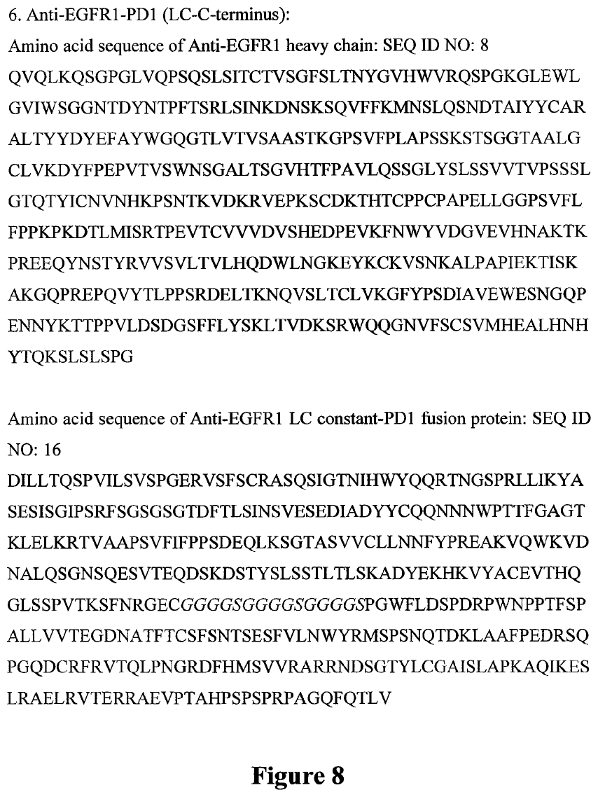

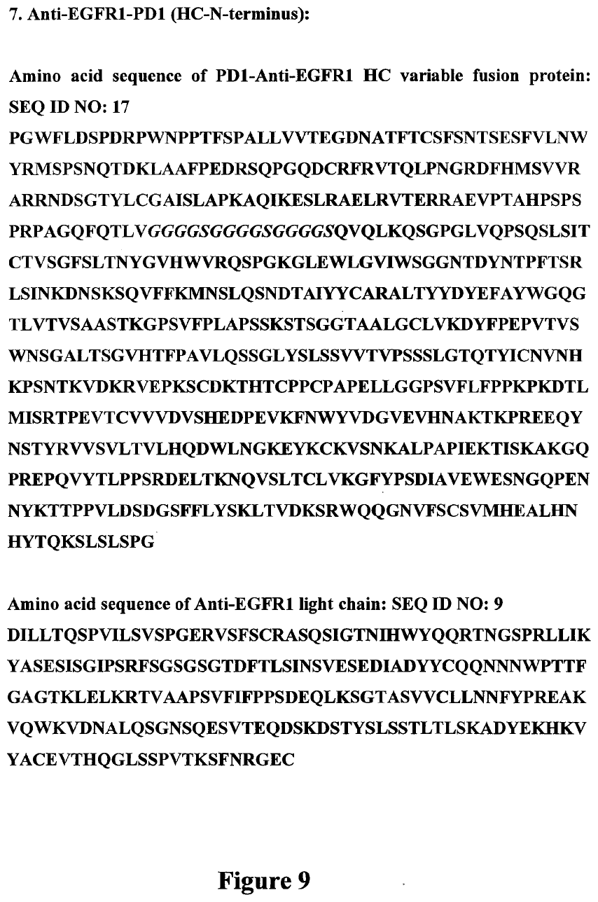

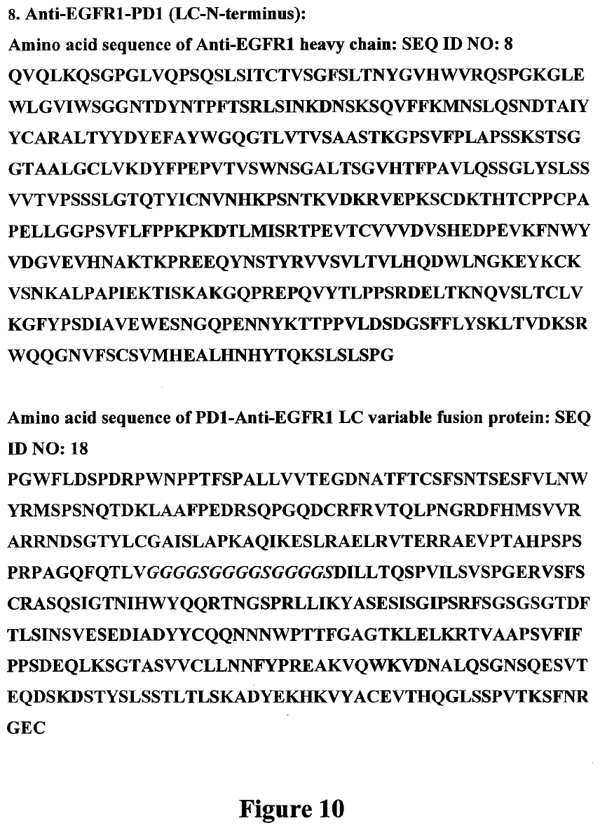

TABLE 1

| Constructs. | |
|---|---|
| no. | Fusion mAbs name |
| FMab5 | Anti-EGFR1 HC-PD1 + Anti-EGFR1 LC |
| FIG. 6 | (AA sequences in FIG. 7, SEQ ID NO: 15 and 9) |
| FMab6 | Anti-EGFR1 HC + Anti-EGFR1 LC-PD1 |
| FIG. 6 | (AA sequences in FIG. 8, SEQ ID NO: 8 and 16) |
| FMab7 | PD1-Anti-EGFR1 HC + Anti-EGFR1 LC |
| FIG. 6 | (AA sequences in FIG. 9, SEQ ID NO: 17 and 9) |
| FMab8 | Anti-EGFR1 HC + PD1-Anti-EGFR1 LC |
| FIG. 6 | (AA sequences in FIG. 10, SEQ ID NO: 8 and 18) |

### Expression of the Above Fusion Constructs in CHO Cells:

The codon-optimized nucleotide sequences of the Anti-EGFR1-PD1 individual domains were optimized for expression in CHO cells. Such optimized sequences (SEQ ID NOs: 1, 2, 7, and 4) were assemble in a mammalian expression vector with help of primers described in Table 2:

TABLE 2

| | |
|---|---|
| FMAB7FP1 | AGA TAT CGC CAC CAT GAT GTC CTT CGT G |
| | SEQ ID NO: 19 |
| FMAB7FP2 | GGC GGC GGA GGC TCT CAG GTG CAG CTG AAG |
| | CAG TC SEQ ID NO: 20 |
| FMAB7RP1 | AGT ATA CTC AGC CGG GGG ACA GAG A |
| | SEQ ID NO: 21 |
| FMAB7RP2 | TTC AGC TGC ACC TGA GAG CCT CCG CCG CCA |
| | CTT C SEQ ID NO: 22 |
| FMAB7LCRP | ATT AAT TAA TCA ACA CTC GCC CCG GTT GAA |
| | GGA CT SEQ ID NO: 23 |
| FMAB6FP2 | CTC TGT CCC CCG GCG GCG GCG GAG GAT CTG |
| | GCG GA SEQ ID NO: 24 |
| FMAB6RP2 | GAT CCT CCG CCG CCG CCG GGG GAC AGA GAC |
| | AGG GA SEQ ID NO: 25 |
| FMAB6RP1 | AGT ATA CTC ACA CCA GGG TCT GGA AC |
| | SEQ ID NO: 26 |

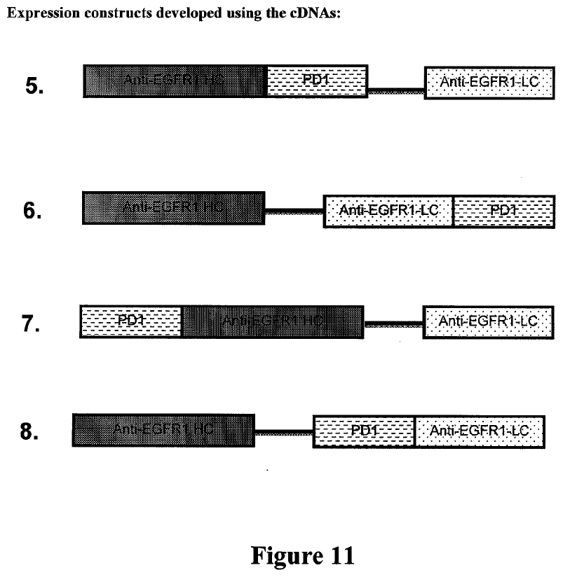

Using the shown above cDNA primers set, constructs were assembled as shown in FIG. 11.

### 2. Anti-EGFR1-TGFβRII Fusion Proteins for Treatment of Cancer

High levels of TGFβ are produced by many types of tumors, including melanomas and cancers of the breast, colon, esophagus, stomach, liver, lung, pancreas, and prostate, as well as hematologic malignancies (see Teicher 2001; Dong 2006). TGFβ is known to be immunosuppressive for T cells and NK cells through blocking of IL-2 and other mechanisms, including generation of T-regs. Several lines of evidence suggest that negating TGFβ activity may enhance anti-tumor effects of T cells (Wrzesinski 2007). Moreover, TGFβ can foster tumor growth through epithelial to mesenchymal transition and promoting angiogenesis. TGFβ expression is also associated with poor prognosis in patients and earlier recurrence. However, considering the pleotropic effects of TGFβ in controlling the immune response, it has been shown that generalized blocking of TGFβ activity may result in widespread auto-inflammatory activity. Hence, localized depletion of TGFβ in the tumor vicinity may be an alternative way to modulate immunosuppressive environment. Anti-EGFR1-TGFβRII fusion protein of the present invention binds to EGFR on the tumor cells and ties up the TGFβ around the tumor to enhance immune response against tumor cells.

### Design and Selection of the Molecules:

The objective is to design the antibody fusion molecules which have duel therapeutic properties. On one hand the molecule should retain the complete activity of the Anti-EGFR1 (cetuximab) and in parallel; it should have the TGFβ binding activity in the tumor environment. The amino acid sequence of the Anti-EGFR1 IgG molecule was retained excepting that the lysine was not expressed at the C-terminus of the heavy chain. Both single and double fusion and expression levels are shown in Table 3, wherein TGFβRII was fused with Anti-EGFR1.

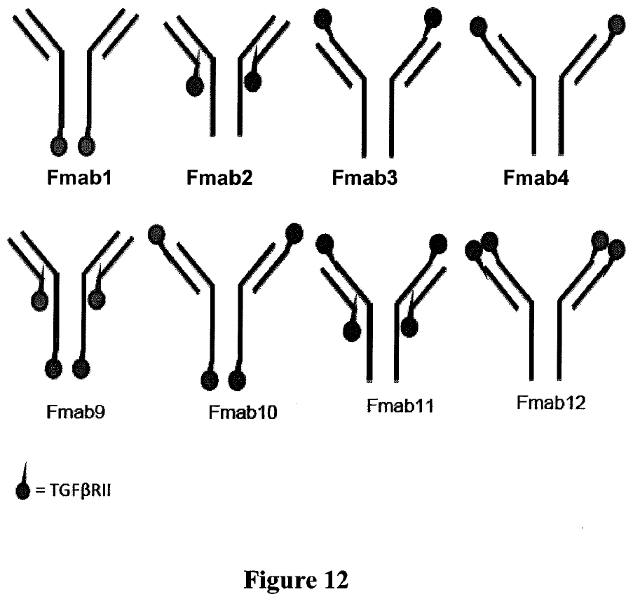

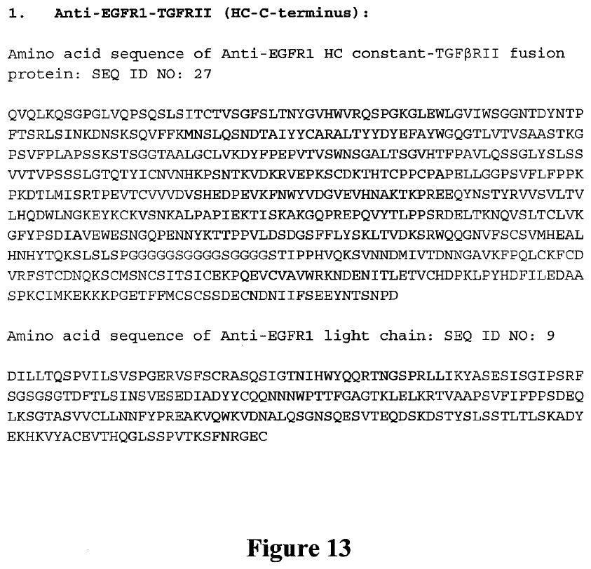

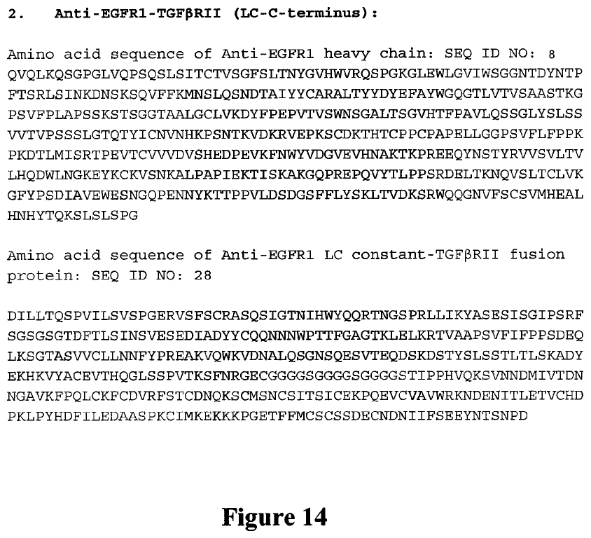

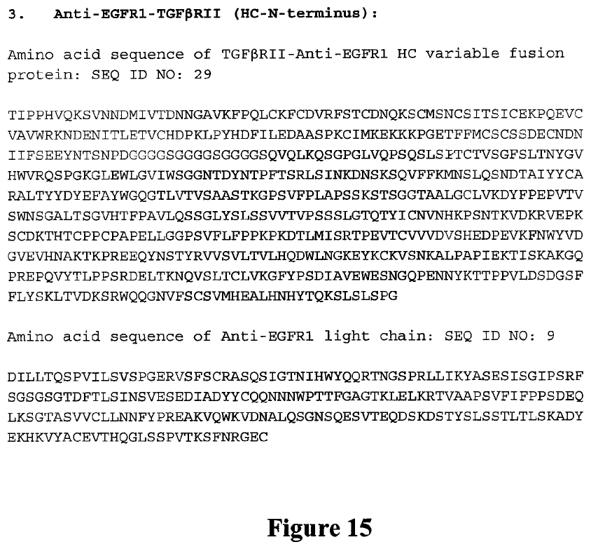

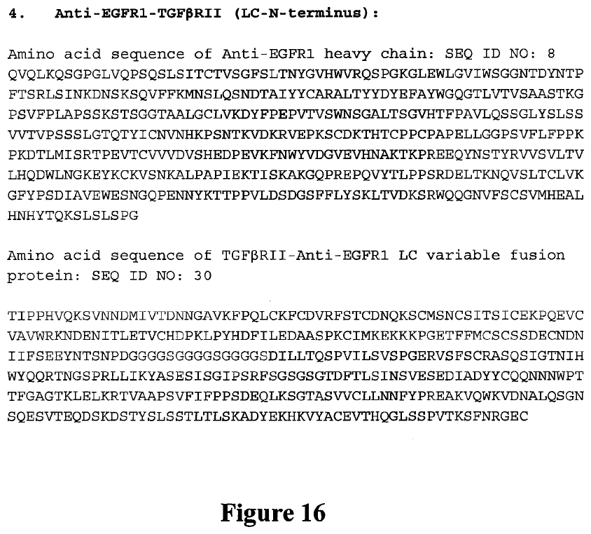

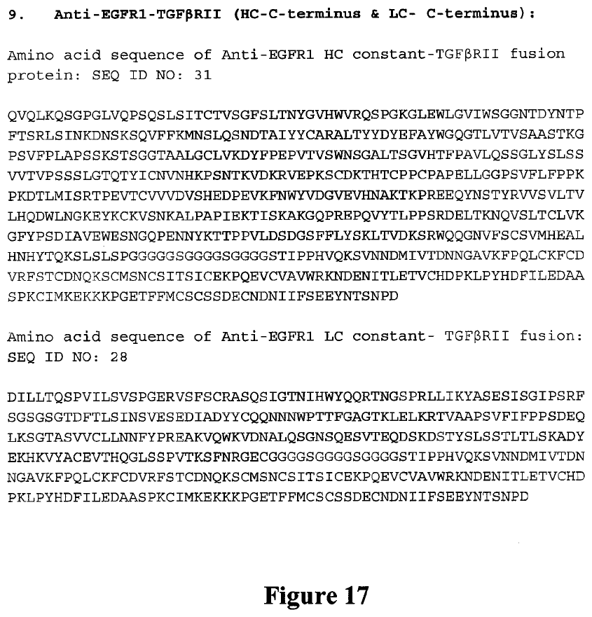

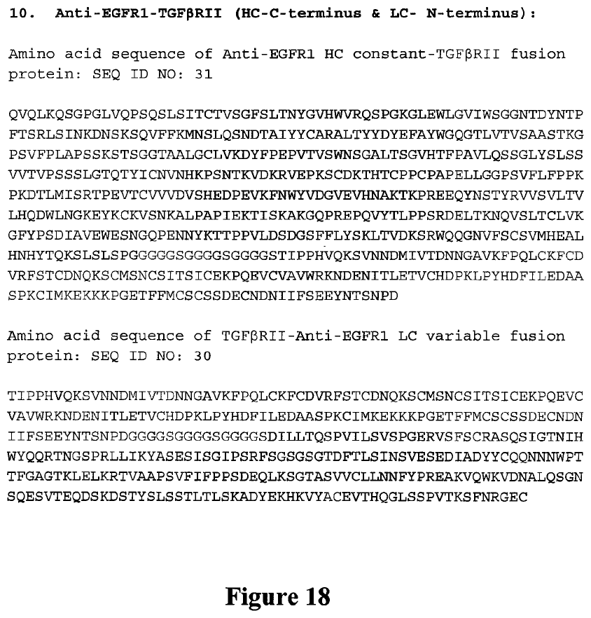

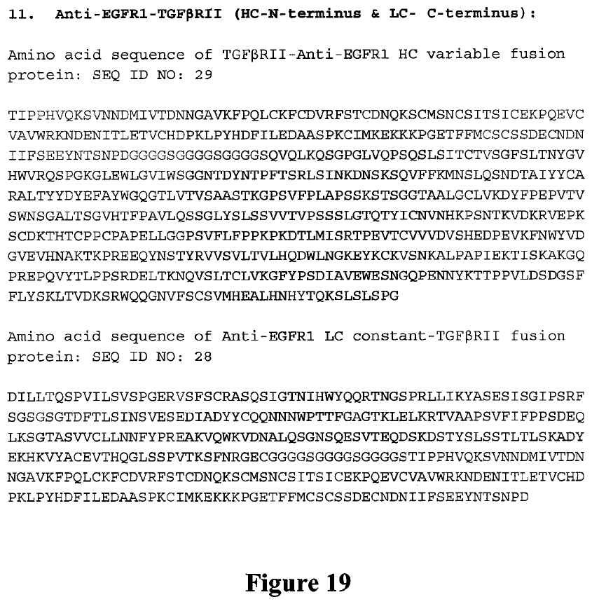

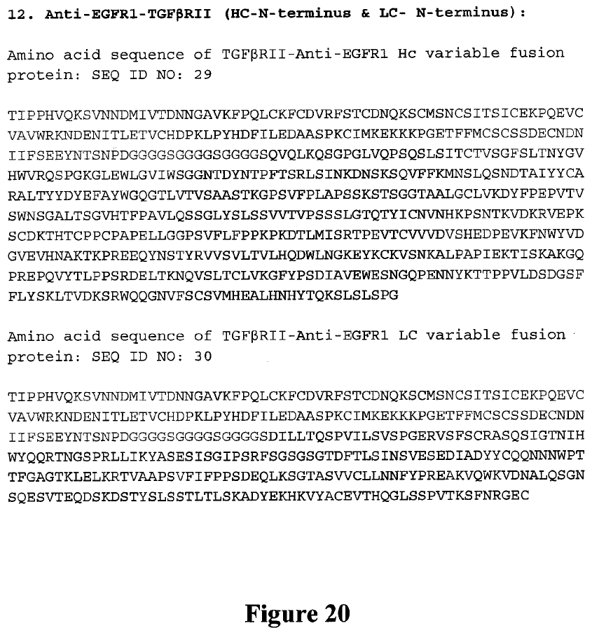

TABLE 3

| Construct. | |
|---|---|
| no. | Fusion Mabs name |
| FMab1 | Anti-EGFR1 HC-TGFβRII + Anti-EGFR1 LC |
| FIG. 12 | (AA sequences in FIG. 13, SEQ ID NO: 27 and 9) |
| FMab2 | Anti-EGFR1 HC + Anti-EGFR1 LC-TGFβRII |
| FIG. 12 | (AA sequences in FIG. 14, SEQ ID NO: 8 and 28) |
| FMab3 | TGFβRII-Anti-EGFR1 HC + Anti-EGFR1 LC |
| FIG. 12 | (AA sequences in FIG. 15, SEQ ID NO: 29 and 9) |
| FMab4 | Anti-EGFR1 HC + TGFβRII-Anti-EGFR1 LC |
| FIG. 12 | (AA sequences in FIG. 16, SEQ ID NO: 8 and 30) |
| FMab9 | Anti-EGFR1 HC-TGFβRII + Anti-EGFR1 LC-TGFβRII) |
| FIG. 12 | (AA sequences in FIG. 17, SEQ ID NO: 31 and 28) |
| FMab10 | Anti-EGFR1 HC-TGFβRII + TGFβRII-Anti-EGFR1 LC |
| FIG. 12 | (AA sequences in FIG. 18, SEQ ID NO: 31 and 30) |
| FMab11 | TGFβRII-Anti-EGFR1 HC + Anti-EGFR1 LC-TGFβRII |
| FIG. 12 | (AA sequences in FIG. 19, SEQ ID NO: 29 and 28) |
| FMab12 | TGFβRII-Anti-EGFR1 HC + TGFβRII-Anti-EGFR1 LC |
| FIG. 12 | (AA sequences in FIG. 20, SEQ ID NO: 29 and 30) |

### Expression of the Above Fusion Constructs in CHO Cells:

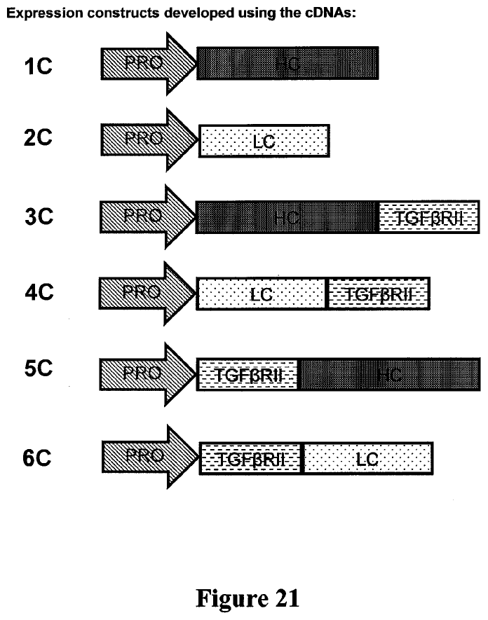

The codon-optimized nucleotide sequences of the Anti-EGFR1-TGFβRII individual domains were optimized for expression in CHO cells. Such sequences (SEQ ID NOs: 1, 2, 4, and 3) were assemble in a mammalian expression vector. The expression constructs are set forth in FIG. 21.

Transfection of the Above Vectors Combination to obtain the Desired Cell Line:

The expression constructs developed above were transfected in the following combination, as set forth in Table 4, into CHO cells to produce the following fusion proteins using the constructs as defined in FIG. 21.

TABLE 4

| | | Expression constructs | Cell line | Titer |
|---|---|---|---|---|
| Sl. No. | Fusion protein Name | combination transfected | used | g/L |
| FMab1 | Anti-EGFR1 HC-TGFβRII + | Expression constructs # | CHO | 0.11 |
| | Anti-EGFR1 LC (HC-C-terminus) | 2 C and 3 C | | |
| FMab2 | Anti-EGFR1 HC + Anti-EGFR1 | Expression constructs # | CHO | 0.10 |
| | LC-TGFβRII | 1 C and 4 C | | |
| FMab3 | TGFβRII-Anti-EGFR1 HC + | Expression constructs # | CHO | 0.09 |
| | Anti-EGFR1 LC | 2 C and 5 C | | |
| FMab4 | Anti-EGFR1 HC + TGFβRII- | Expression constructs # | CHO | 0.08 |
| | Anti-EGFR1 LC | 1 C and 6 C | | |
| FMab9 | Anti-EGFR1 HC-TGFβRII + | Expression constructs # | CHO | ND/VL |
| | Anti-EGFR1 LC-TGFβRII | 3 C and 4 C | | |
| FMab10 | Anti-EGFR1 HC-TGFβRII + | Expression constructs # | CHO | 0.06 |
| | TGFβRII-Anti-EGFR1 LC | 3 C and 6 C | | |
| FMab11 | TGFβRII-Anti-EGFR1 HC + | Expression constructs # | CHO | ND/VL |
| | Anti-EGFR1 LC-TGFβRII | 4 C and 5 C | | |
| FMab12 | TGFβRII-Anti-EGFR1 HC + | Expression constructs # | CHO | 0.06 |
| | TGFβRII-Anti-EGFR1 LC | 5 C and 6 C | | |

### Purification of the Fusion Mabs Supernatants Using Protein A Column:

The fusion monoclonal antibodies (Mabs) using recombinant protein producing CHO cell culture supernatant.

### Procedure:

The procedure describes in detail the small scale purification process of IgG using C10/10 or XK26 column and using Mab Select Xtra affinity resin. The samples generated by this protocol can be used for various analysis

### Process Flow:

The culture supernatant secreted from recombinant cell line producing monoclonal antibodies or fusion monoclonal antibodies under sterile conditions were tested for titer and endotoxins;
The affinity chromatography using Mab Select Xtra Protein A resin was washed and equilibrated with binding buffer;
The pH of the supernatant was adjusted using 0.5M phosphate to the same pH has the column;
The supernatant was allowed to bind to the column/pass through the column at the flow rate of 0.5 ml/minute to achieve the maximum binding;
All the fusion Mabs binds through the Fc region and rest of the impurities passed pass through as flow through;
The column was washed with equilibration buffer;
The bound fusion Mabs were eluted using 0.1 M glycine pH 3.0;
The eluted proteins were adjusted back to neutral pH or the stable formulation pH;
The purified proteins were stored at −20° C. or at 2-8° C. depending on the stability.

### Analysis of Protein A Purified Fusion Mabs Using SDS PAGE:

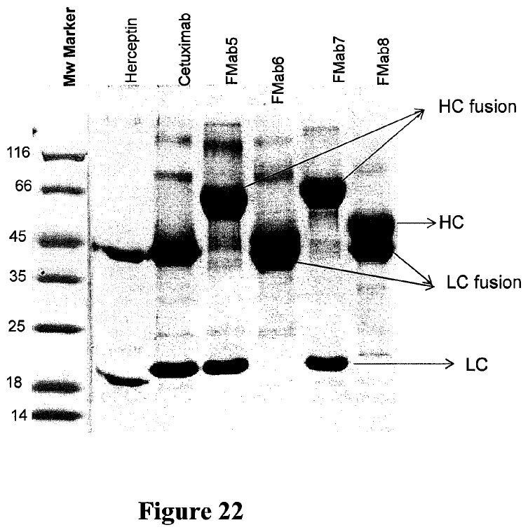

The transfected supernatants obtained were purified using proteinA affinity column. Later these were analyzed on reducing and non-reducing SDS-PAGE to find out the integrity of the molecule, as shown in FIG. 22, where in the proteinA purified samples were analyzed on 12% reducing SDS-PAGE. As expected all the fusion partners are giving the expected pattern on SDS-PAGE. The LC fusion and HC are running closely but the bands are separated. This higher mobility may be due to the 8 N-glycosylation sites (TGBRII 3*2-6+2 on LC)

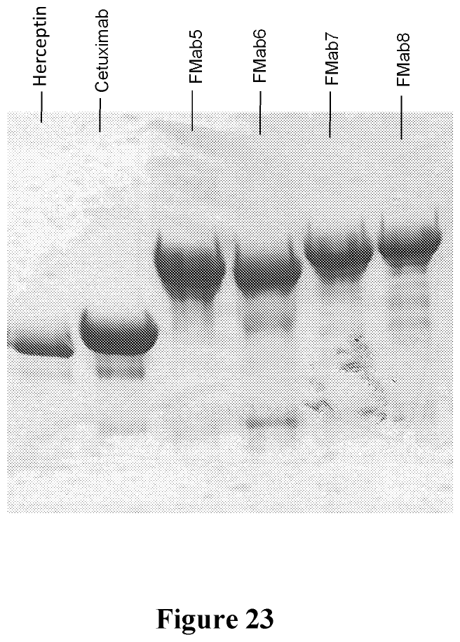

FIG. 23 shows the results of the ProteinA purified samples that were analyzed on 6% non-reducing SDS-PAGE and although the amino acid composition is same, there is a difference in mobility. It may be due to the variable levels of glycosylation pattern based on the TGFβRII position and access in the molecule.

### 3. Anti-CTLA4-PD1 Fusion Protein Constructs for Cancer Targets

Immunohistochemical analysis of freshly isolated tumor samples from patients with ovarian, lung, and breast cancers, renal cell carcinoma, squamous cell carcinoma of the head and neck, esophageal carcinoma, glioblastoma, thymoma, colon carcinoma, pancreatic and melanoma found that the vast majority express B7-H1 (see Flies 2011; Nomi 2007). Several pre-clinical studies have demonstrated increased tumor rejection by blocking PD1-PDL1 interaction. Recently, anti-PD1 and PD-L1 based therapies have demonstrated considerable activity in melanoma and some other solid tumors confirming their application as one of the most promising anti-cancer therapies.

Although, anti-CTLA4 may allow co-stimulation of T cells, they may still be inhibited by PD-L1-PD-1 interaction. This may be one of the reasons for only a minority of patients having response to anti-CTLA4 antibody. Fusion antibody of both anti-CTLA4 and PD1 are more efficacious than either agent alone since anti-CTLA4 allows T cell co-stimulation whereas PD1 binds to PD-L1 on tumor cells to negate the immunosuppression of T cells in tumor microenvironment. This may even be safer than the anti-CTLA4 because the lone use of anti-CTLA4 has led to immune breakthrough adverse events.

### Design and Selection of the Molecules:

The objective is to design the antibody fusion molecules which have duel therapeutic properties. On one hand the molecule should retain the complete activity of the anti-CTLA4 (Ipilimumab) and in parallel; it should have the PD L 1 receptor binding activity in the tumor environment. The complete amino acid sequence of the anti-CTLA4 IgG molecule was used except the removal of the lysine at the C-terminus of the heavy chain. A 15 amino acid linker was positioned between the PD1 and Anti-CTLA4. The following combinations of constructs, as set forth in Table 5, were designed as shown in FIG. 24. The details of the above fusion protein constructs are given below.

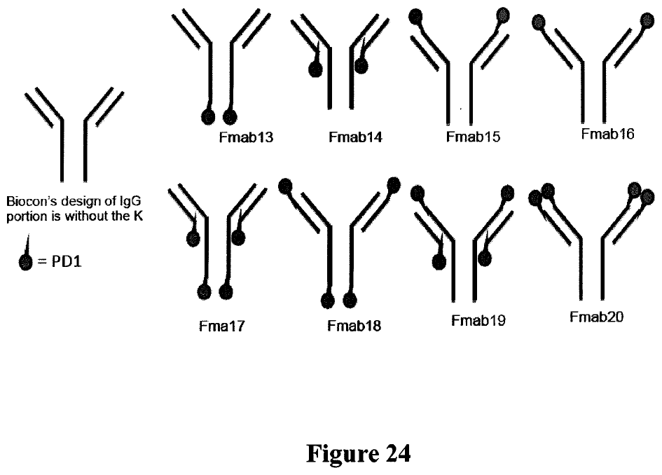

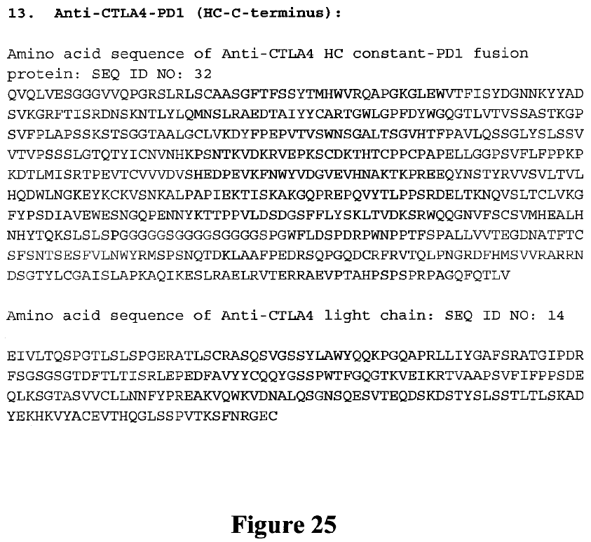

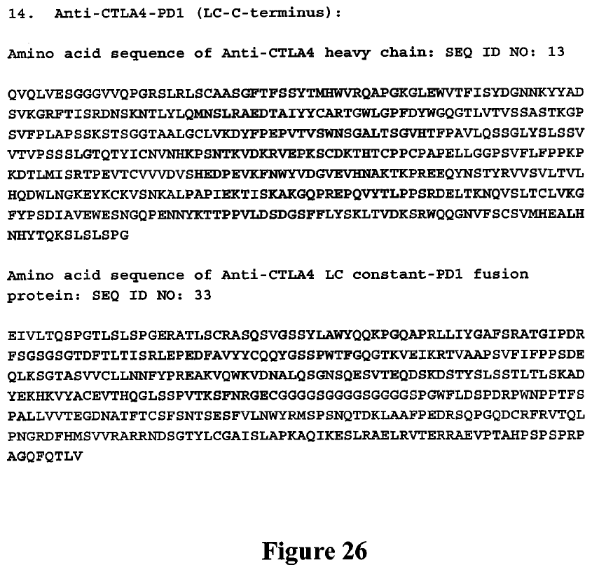

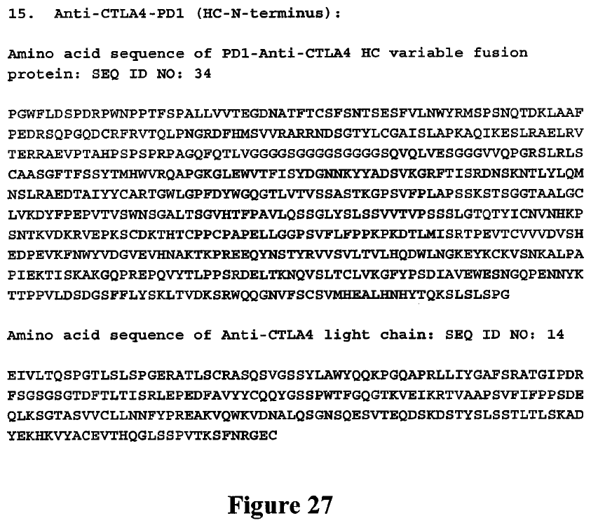

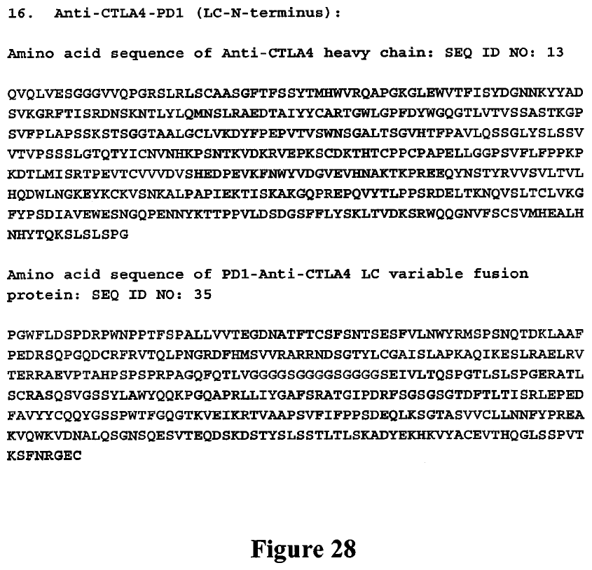

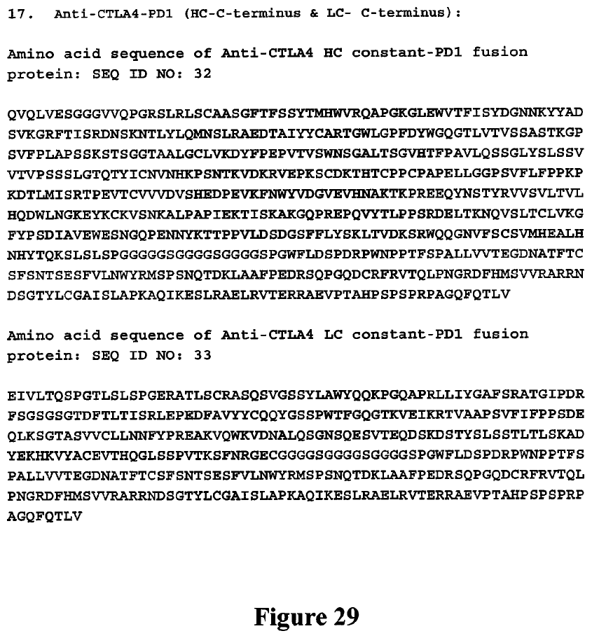

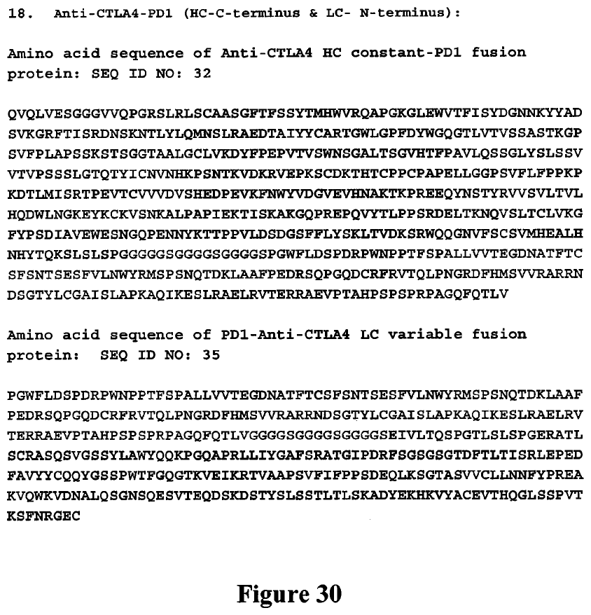

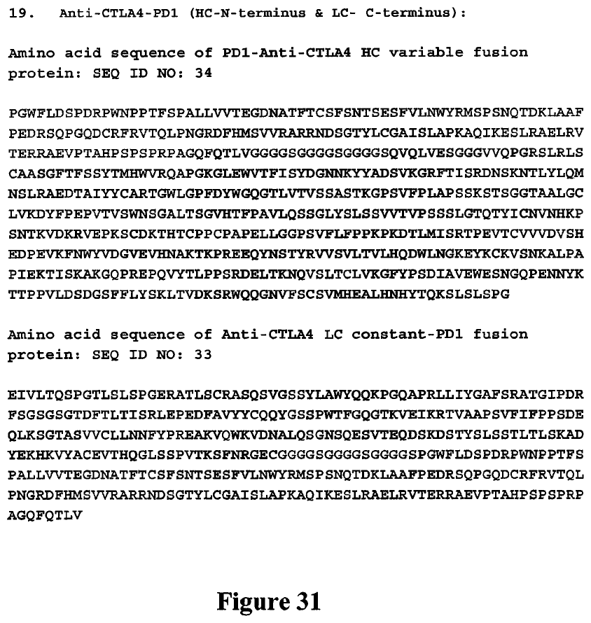

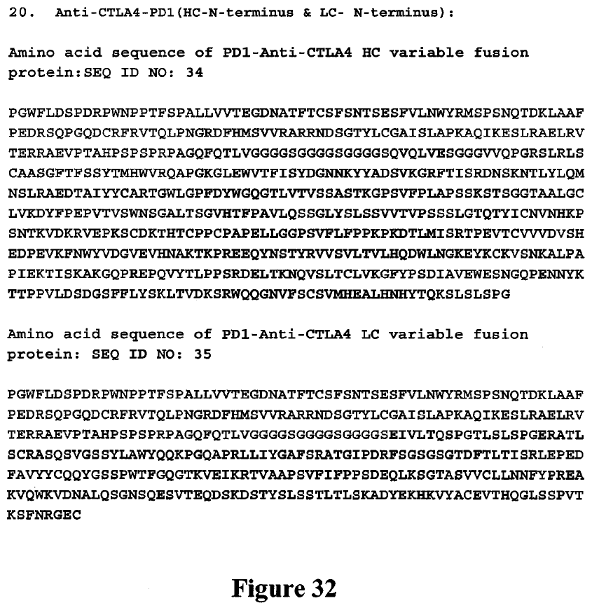

TABLE 5

| Constructs. | |
|---|---|
| no. | Fusion Mabs name |
| FMab13 | Anti-CTLA4 HC-PD1 + Anti-CTLA4 LC |
| FIG. 21 | (AA sequences in FIG. 25, SEQ ID NO: 32 and 14) |
| FMab14 | Anti-CTLA4 HC + Anti-CTLA4 LC-PD1 |
| FIG. 21 | (AA sequences in FIG. 26, SEQ ID NO: 13 and 33) |
| FMab15 | PD1-Anti-CTLA4 HC + Anti-CTLA4 LC |
| FIG. 21 | (AA sequences in FIG. 27, SEQ ID NO: 34 and 14) |
| FMab16 | Anti-CTLA4 HC + PD1-Anti-CTLA4 LC |
| FIG. 21 | (AA sequences in FIG. 28, SEQ ID NO: 13 and 35) |
| FMab17 | Anti-CTLA4 HC-PD1 + Anti-CTLA4 LC-PD1 |
| FIG. 21 | (AA sequences in FIG. 29, SEQ ID NO: 32 and 33) |
| FMab18 | Anti-CTLA4 HC-PD1 + PD1-Anti-CTLA4 LC |
| FIG. 21 | (AA sequences in FIG. 30, SEQ ID NO: 32 and 35) |
| FMab19 | PD1-Anti-CTLA4 HC + Anti-CTLA4 LC-PD1 |
| FIG. 21 | (AA sequences in FIG. 31, SEQ ID NO: 34 and 33) |
| FMab20 | PD1-Anti-CTLA4 HC + PD1-Anti-CTLA4 LC |
| FIG. 21 | (AA sequences in FIG. 32, SEQ ID NO: 34 and 35) |

### Expression of the Above Fusion Constructs in CHO Cells:

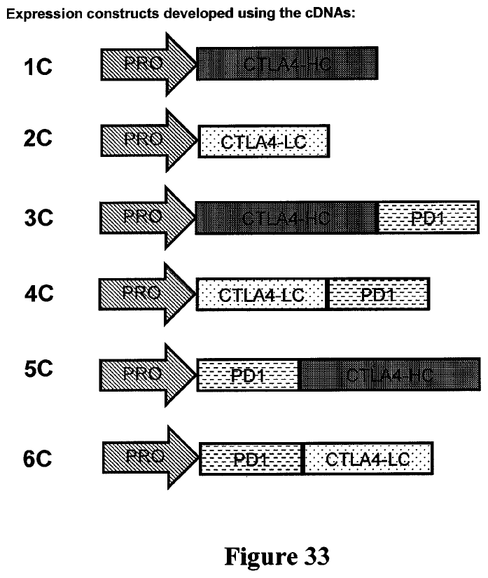

The complete nucleotide sequence of the Anti-CTLA4-PD1 individual domains were codon optimized for expression in CHO cells (SEQ ID NOs: 7, 4, 5 and 6). The cDNAs were synthesized. The constructs were assembled in mammalian expression vectors. The expression of anti-CTLA4-PD1 fusion proteins using the constructs as set forth in FIG. 33.

The expression constructs developed and shown in FIG. 33 were transfected in the following combination into CHO cells (Table 6) to produce the following fusion proteins. The titer obtained for each constructs are mentioned in the last column.

TABLE 6

| | | Expression constructs | Cell line | Titer |
|---|---|---|---|---|
| Sl. No. | Fusion protein Name | combination transfected | used | g/L |
| FMab | Anti-CTLA4 HC-PD1 + | Expression constructs # | CHO | 0.175 |
| 13 | Anti-CTLA4 LC | 2 C and 3 C | | |
| FMab | Anti-CTLA4 HC + | Expression constructs # | CHO | 0.221 |
| 14 | Anti-CTLA4 LC-PD1 | 1 C and 4 C | | |
| FMab | PD1-Anti-CTLA4 HC + | Expression constructs # | CHO | 0.029 |
| 15 | Anti-CTLA4 LC | 2 C and 5 C | | |
| FMab | Anti-CTLA4 HC + PD1- | Expression constructs # | CHO | 0.021 |
| 16 | Anti-CTLA4 LC | 1 C and 6 C | | |
| FMab | Anti-CTLA4 HC-PD1 + | Expression constructs # | CHO | 0.137 |
| 17 | Anti-CTLA4 LC-PD1 | 3 C and 4 C | | |
| FMab | Anti-CTLA4 HC-PD1 + | Expression constructs # | CHO | 0.012 |
| 18 | PD1-Anti-CTLA4 LC | 3 C and 6 C | | |
| FMab | PD1-Anti-CTLA4 HC + | Expression constructs # | CHO | 0.029 |
| 19 | Anti-CTLA4 LC-PD1 | 4 C and 5 C | | |
| FMab | PD1-Anti-CTLA4 HC + | Expression constructs # | CHO | 0.014 |
| 20 | PD1-Anti-CTLA4 LC | 5 C and 6 C | | |

### Purification of and Characterization of Fusion Proteins:

The procedure describes the use of small scale purification process of IgG using C10/10 or XK26 column and using Mab Select Xtra affinity resin. The samples generated by this protocol can be used for various analysis.

### Process Flow:

The culture supernatant secreted from recombinant cell line producing monoclonal antibodies or fusion monoclonal antibodies under sterile conditions were tested for titer and endotoxins;
The affinity chromatography using Mab Select Xtra ProteinA resin was washed and equilibrated with binding buffer;
The pH of the supernatant was adjusted using 0.5M phosphate to the same PH has the column;
The supernatant was allowed to bind to the column/pass through the column at the flow rate of 0.5 ml/minute to achieve the maximum binding;
All the fusion Mabs binds through the Fc region and rest of the impurities passed through as flow through;
The column was washed with equilibration buffer;
The bound fusion Mabs were eluted using 0.1 M glycine pH 3.0;
The eluted proteins were adjusted back to neutral pH or the stable formulation pH; and
The purified proteins are stored at −20° C. or at 2-8° C. depending on the stability.

### 4. Anti-EGFR1 HC-TGFβRII+Anti-EGFR1 LC (Fmab 1)

### Binding ELISAs-Procedure:

The fusion Mab was tested for its ability to bind to its targets in three different ELISAs: 1) EGFR1 target-binding ELISA, 2) TGFβ-target binding ELISA and 3) Bifunctional ELISA.

For the target binding ELISAs, the targets (rhEGFR-Fc chimera or TGFβ) were coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. Binding of the fusion Mab was detected by the addition of a biotinylated anti-human IgG F(ab)₂ secondary antibody, followed by a 1 hr incubation with peroxidase—conjugated streptavidin at room temperature. TMB substrate solution was added and the reaction stopped with IN H₂SO₄. The absorbance was measured at 450 nm on a BioTek Synergy H4 Hybrid reader.

For the bifunctional ELISA, rhEGFR-Fc chimera was coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. After washing, TGFβ was added and the plate was incubated at room temperature for 1 hr. The plate was washed and anti-TGFβ-biotin was added and the plate incubated at room temperature for 1 hr. The plate was washed and streptavidin-HRP was added and the plate incubated at room temperature for 1 hr. After washing, TMB substrate solution was added and the reaction stopped with IN H₂SO₄. The absorbance was measured at 450 nm on a BioTek Synergy H4 Hybrid reader.

### Results:

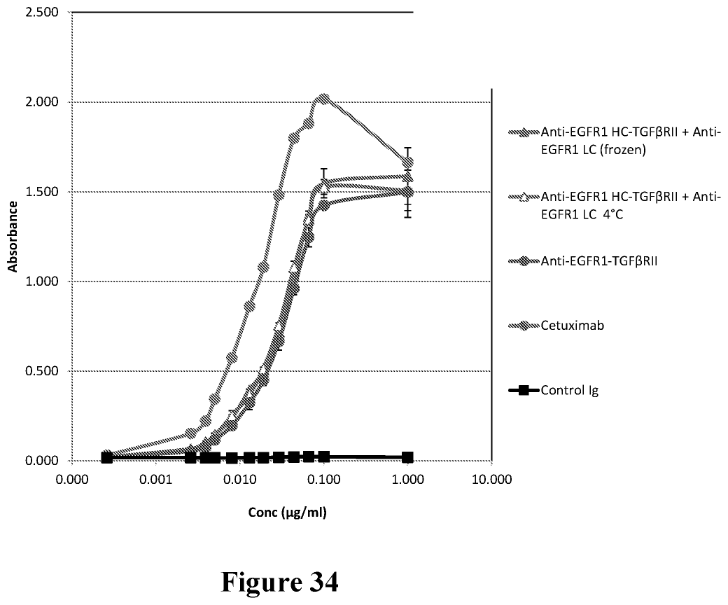

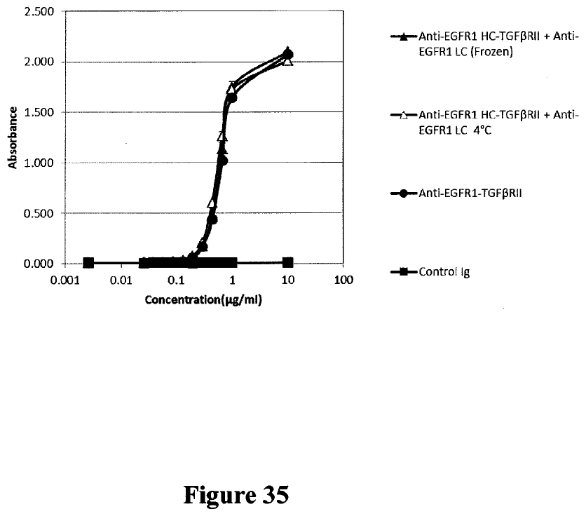

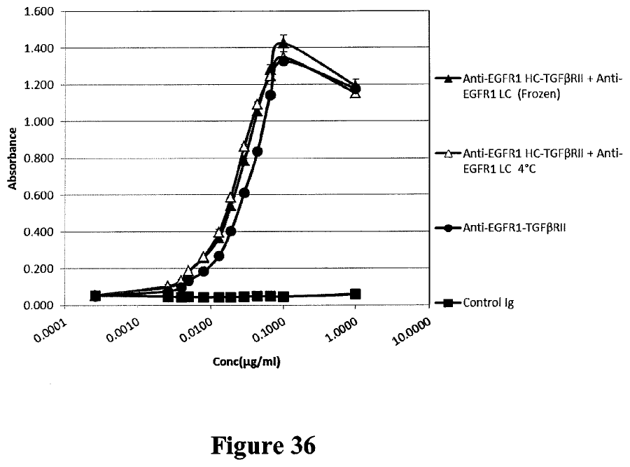

The binding of the anti-EGFR1 HC-TGFβRII+Anti-EGFR1 LC fusion Mab to both the targets EGFR1 (FIG. 34) and TGFβ (FIG. 35) was comparable with anti-EGFR1-TGFβRII. The anti-EGFR1 HC-TGFβRII+Anti-EGFR1 LC fusion Mab was also tested in a bifunctional ELISA to determine whether the anti-EGFR1 and TGFβRII domains of the Mab can bind to their respective targets without interfering with each other. As seen in FIG. 36, the anti-EGFR1 HC-TGFβRII+Anti-EGFR1 LC fusion Mab binds to both its targets, suggesting that there is no interference in binding to either target dues to the construction of the fusion Mab.

### Binding to Cells Expressing EGFR1

### Procedure:

A-431 cells were grown in flasks until they reached 70-80% confluency. The cells were trypsinized and harvested. The cells were stained with different dilutions of the anti-EGFR1 HC-TGFβRII+Anti-EGFR1 LC fusion Mab or control Ig at 2-8° C. for 30 minutes. The cells were washed and incubated with anti-Human IgG-FITC conjugate at 2-8° C. for 30 minutes. After washing, the cells were analyzed on a flow cytometer. Live cells were gated based on their FSC vs SSC profiles. The total MFI for the gated population were recorded.

### Results:

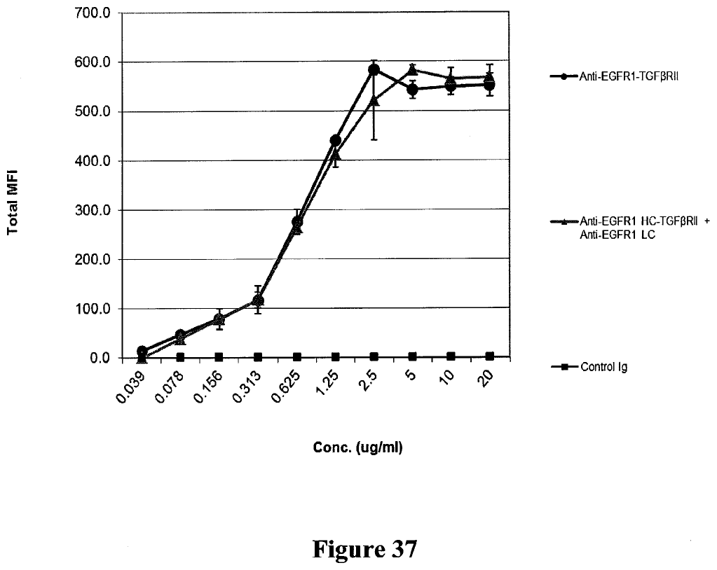

The anti-EGFR1 HC-TGFβRII+Anti-EGFR1 LC fusion binds to EGFR-expressing A-431 cells in a dose dependent manner (FIG. 37). The binding of anti-EGFR1 HC-TGFβRII+Anti-EGFR1 LC is comparable to the binding of anti-EGFR1-TGFβRII.

### Antibody-Dependent Cytotoxicity Activity

### Procedure:

A-431 cells were grown in flasks until they reached 70-80% confluency. The cells were trypsinized, harvested and plated into 96-well plates. The cells were labeled with different dilutions of the anti-EGFR1 HC-TGFβRII+Anti-EGFR1 LC fusion Mab or control Ig at 2-8° C. for 30 minutes. The labeled cells were co-incubated with freshly isolated human PBMC at 37° C., 5% CO₂ for 24 hours. Cytotoxicity was measured using the Cyto-Tox-Glo cytotoxicity assay kits.

### Results:

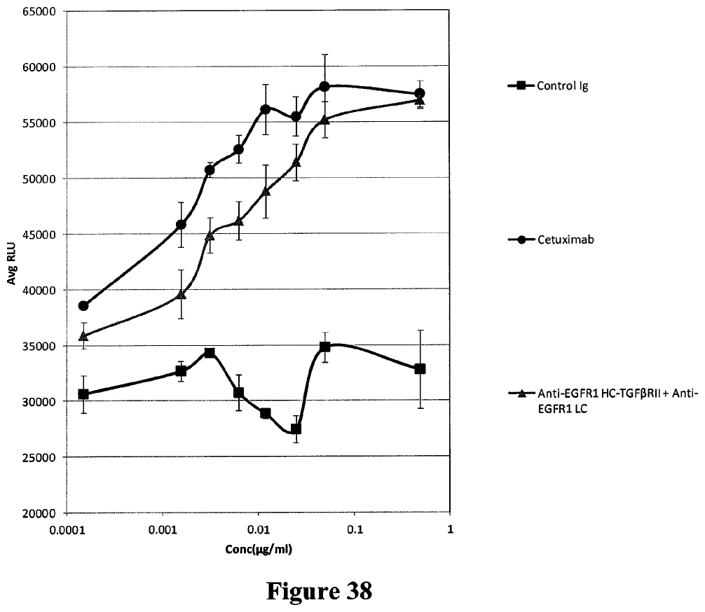

The anti-EGFR1 HC-TGFβRII+Anti-EGFR1 LC fusion mediates ADCC of EGFR-expressing A-431 cells by human PBMC effector cells. The ADCC is dose dependent (FIG. 38). These results suggest that the Fc portion of the fusion Mab is intact and functional.

### Inhibition of Proliferation

### Procedure:

A-431 cells were grown in flasks until they reached 70-80% confluency. The cells were trypsinized, harvested and plated into 96-well plates. Different dilutions of the anti-EGFR1 HC-TGFβRII+Anti-EGFR1 LC fusion Mab or control Ig were added to the cells. The plates were incubated at 37° C., 5% CO₂ for three days. On the third day, cell proliferation was measured by the AlamarBlue method.

### Results:

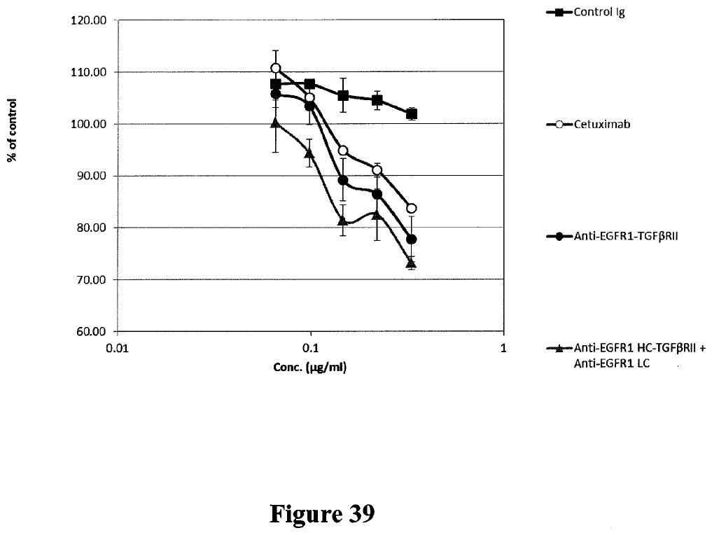

Anti-EGFR antibodies such as Cetuximab are known to inhibit the proliferation of EGFR1-expressing cells. As seen in FIG. 39, the anti-EGFR portion of the anti-EGFR1 HC-TGFβRII+Anti-EGFR1 LC fusion Mab is intact and has anti-proliferative activity.

### 5. Anti-EGFR1 HC+Anti-EGFR1 LC-TGFβRII (Fmab2)

### Binding ELISAs: Procedure:

The anti-EGFR1 HC+Anti-EGFR1 LC-TGFβRII fusion Mab was tested for its ability to bind to its targets in three different ELISAs: 1) EGFR1 target-binding ELISA, 2) TGF (3-target binding ELISA and 3) Bifunctional ELISA.

For the target binding ELISAs, the targets (rhEGFR-Fc chimera or TGF (3) were coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. Binding of the fusion Mab was detected by the addition of a biotinylated anti-human IgG F(ab)₂ secondary antibody, followed by a 1 hr incubation with peroxidase—conjugated streptavidin at room temperature. TMB substrate solution was added and the reaction stopped with IN H₂SO₄. The absorbance was measured at 450 nm on a BioTek Synergy H4 Hybrid reader.

For the bifunctional ELISA, rhEGFR-Fc chimera was coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. After washing, TGFβ was added and the plate is incubated at room temperature for 1 hr. The plate was washed and anti-TGFβ-biotin was added and the plate incubated at room temperature for 1 hr. The plate was washed and streptavidin-HRP was added and the plate incubated at room temperature for 1 hr. After washing, TMB substrate solution was added and the reaction stopped with 1N H₂SO₄. The absorbance was measured at 450 nm on a BioTek Synergy H4 Hybrid reader.

### Results:

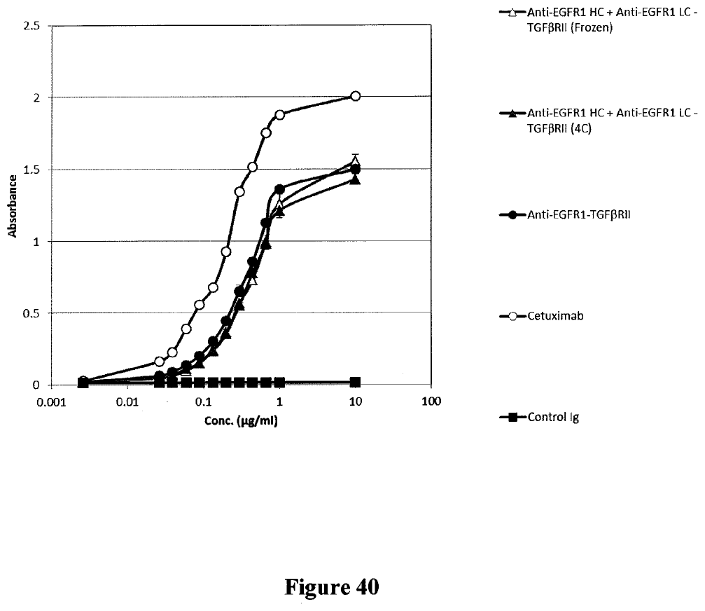

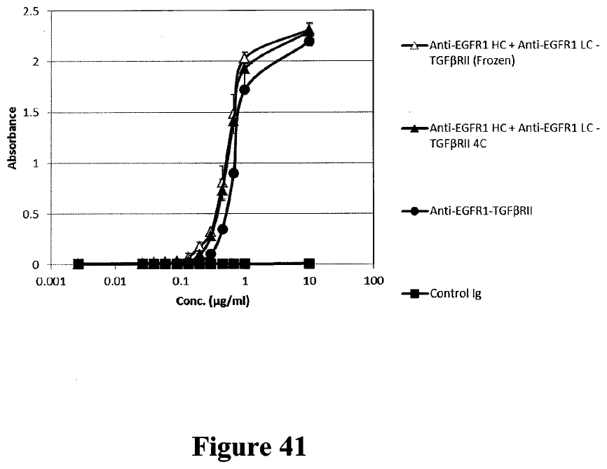

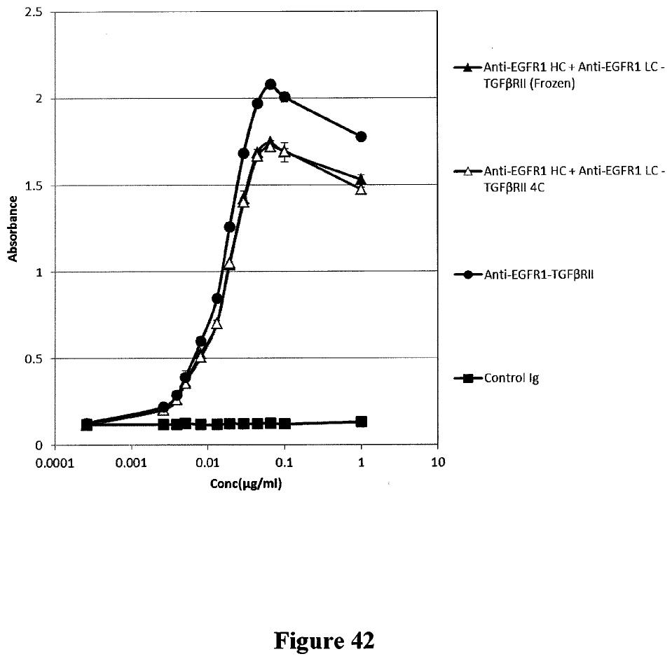

The binding of anti-EGFR1 HC+Anti-EGFR1 LC-TGFβRII fusion Mab to both the targets EGFR1 (FIG. 40) and TGFβ (FIG. 41) was comparable to anti-EGFR1-TGFβRII. The anti-EGFR1 HC+Anti-EGFR1 LC-TGFβRII fusion Mab was also tested in a bifunctional ELISA to determine whether the anti-EGFR1 and TGFβRII domains of the Mab can bind to their respective targets without interfering with each other. As seen in FIG. 42, the anti-EGFR1 HC+Anti-EGFR1 LC-TGFβRII fusion Mab binds to both its targets, suggesting that there is no interference in binding to either target dues to the construction of the fusion Mab.

### Inhibition of Proliferation

### Procedure:

A-431 cells were grown in flasks until they reached 70-80% confluency. The cells were trypsinized, harvested and plated into 96-well plates. Different dilutions of the anti-EGFR1 HC+Anti-EGFR1 LC-TGFβRII fusion Mab or control Ig were added to the cells. The plates were incubated at 37° C., 5% CO2 for three days. On the third day, cell proliferation was measured by the AlamarBlue method.

### Results:

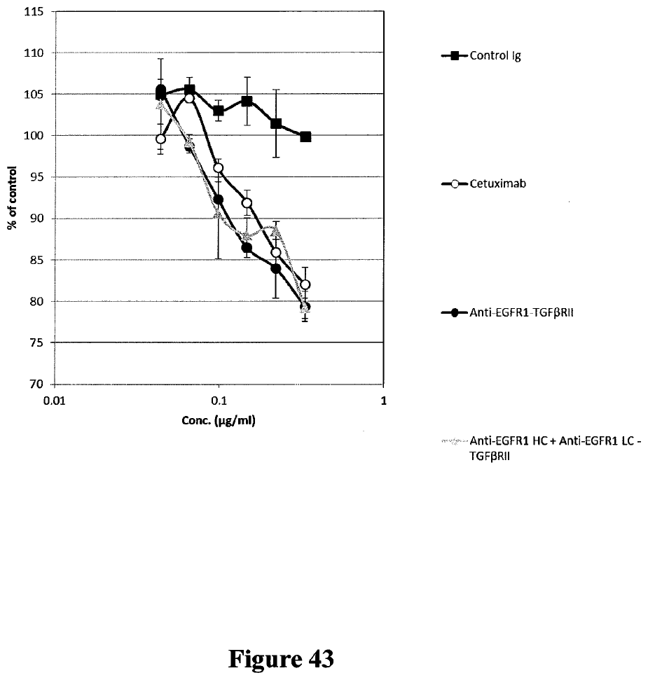

Anti-EGFR antibodies such as Cetuximab are known to inhibit the proliferation of EGFR1-expressing cells. As seen in FIG. 43, the anti-EGFR portion of the anti-EGFR1 HC+Anti-EGFR1 LC-TGFβRII fusion Mab is intact and has anti-proliferative activity.

### 6. TGFβRII-Anti-EGFR1 HC+Anti-EGFR1 LC (Fmab 3)

### Binding ELISAs: Procedure:

The fusion Mab was tested for its ability to bind to its targets in three different ELISAs: 1) EGFR1 target-binding ELISA, 2) TGFβ-target binding ELISA and 3) Bifunctional ELISA.

For the target binding ELISAs, the targets (rhEGFR-Fc chimera or TGFβ) were coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. Binding of the fusion Mab was detected by the addition of a biotinylated anti-human IgG F(ab)₂ secondary antibody, followed by a 1 hr incubation with peroxidase-conjugated streptavidin at room temperature. TMB substrate solution was added and the reaction stopped with IN H₂SO₄. The absorbance was measured at 450 nm on a BioTek Synergy H4 Hybrid reader.

For the bifunctional ELISA, rhEGFR-Fc chimera was coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. After washing, TGFβ was added and the plate was incubated at room temperature for 1 hr. The plate was washed and anti-TGFβ-biotin was added and the plate incubated at room temperature for 1 hr. The plate was washed and streptavidin-HRP was added and the plate incubated at room temperature for 1 hr. After washing, TMB substrate solution was added and the reaction stopped with IN H₂SO₄. The absorbance was measured at 450 nm on a BioTek Synergy H4 hybrid reader.

### Results:

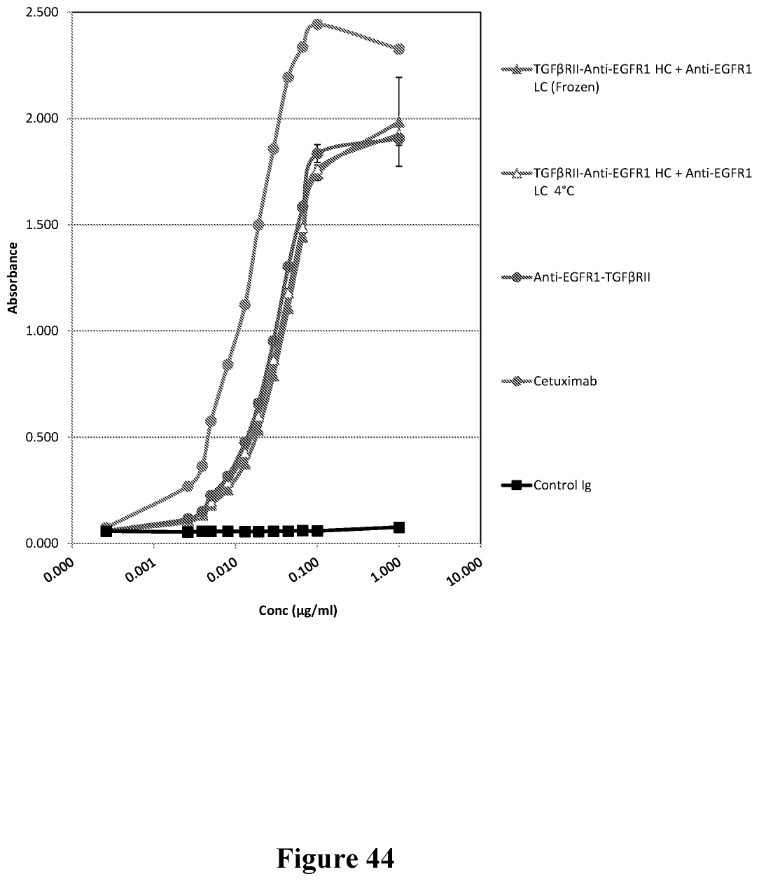

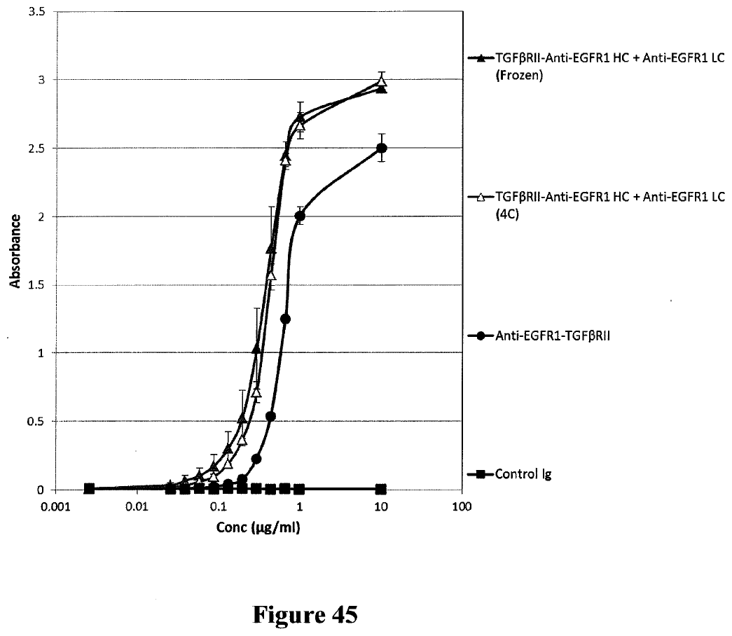

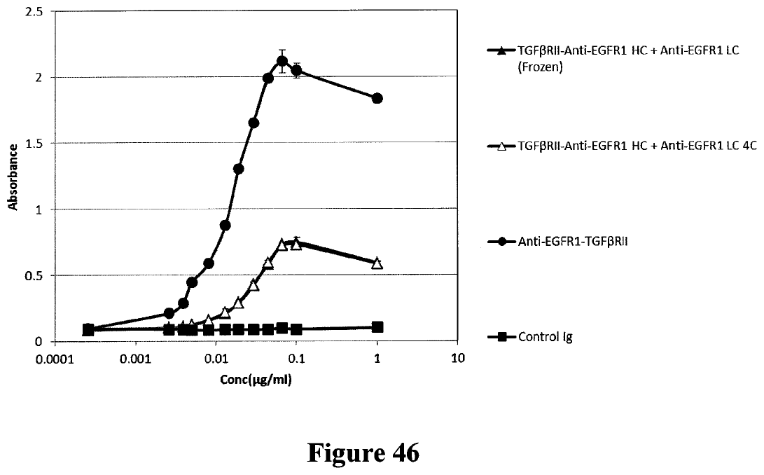

The binding of the TGFβRII-Anti-EGFR1 HC+Anti-EGFR1 LC fusion Mab to both the targets EGFR1 (FIG. 44) and TGFβ (FIG. 45) was comparable to anti-EGFR1-TGFβRII. The TGFβRII-Anti-EGFR1 HC+Anti-EGFR1 LC fusion Mab was also tested in a bifunctional ELISA to determine whether the anti-EGFR1 and TGFβRII domains of the Mab can bind to their respective targets without interfering with each other. As seen in FIG. 46, the binding of TGFβRII-Anti-EGFR1 HC+Anti-EGFR1 LC fusion Mab is reduced as compared to anti-EGFR1-TGFβRII, suggesting that there is some interference in binding to either target due to the construction of the fusion Mab.

### Binding to Cells Expressing EGFR1

### Procedure:

A-431 cells were grown in flasks until they reached 70-80% confluency. The cells were trypsinized and harvested. The cells were stained with different dilutions of the TGFβRII-Anti-EGFR1 HC+Anti-EGFR1 LC fusion Mab or control Ig at 2-8° C. for 30 minutes. The cells were washed and incubated with anti-Human IgG-FITC conjugate at 2-8° C. for 30 minutes. After washing, the cells were analyzed on a flow cytometer. Live cells were gated based on their FSC vs SSC profiles. The total MFI for the gated population were recorded.

### Results:

The TGFβRII-Anti-EGFR1 HC+Anti-EGFR1 LC fusion binds to EGFR-expressing A-431 cells in a dose dependent manner. The binding of TGFβRII-Anti-EGFR1 HC+Anti-EGFR1 LC is comparable to the binding of anti-EGFR1-TGFβRII.

### Inhibition of Proliferation

### Procedure:

A-431 cells were grown in flasks until they reached 70-80% confluency. The cells were trypsinized, harvested and plated into 96-well plates. Different dilutions of the TGFβRII-Anti-EGFR1 HC+Anti-EGFR1 LC fusion Mab or control Ig were added to the cells. The plates were incubated at 37° C., 5% CO₂ for three days. On the third day, cell proliferation was measured by the AlamarBlue method.

### Results:

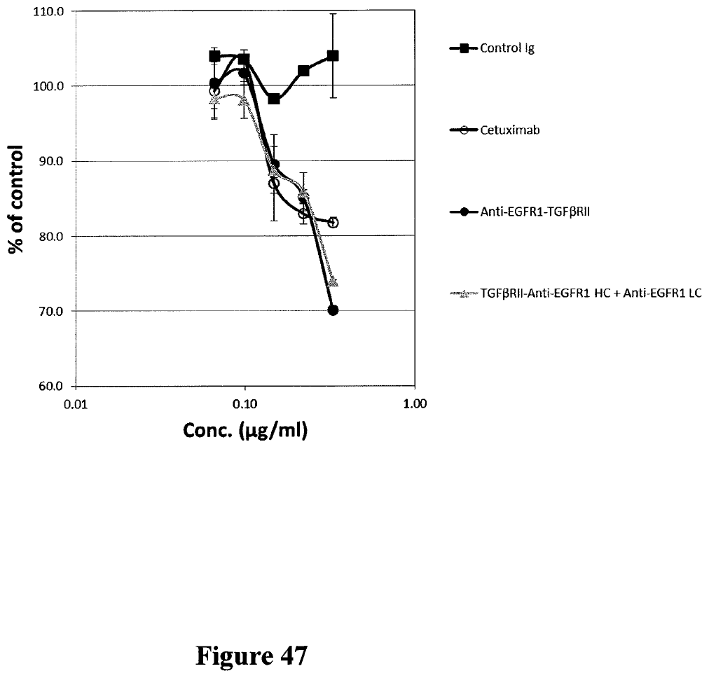

Anti-EGFR antibodies such as Cetuximab are known to inhibit the proliferation of EGFR1-expressing cells. As seen in FIG. 47, the anti-EGFR portion of the TGFβRII-Anti-EGFR1 HC+Anti-EGFR1 LC fusion Mab is intact and has anti-proliferative activity.

### 7. Anti-EGFR1 HC+TGFβRII-Anti-EGFR1 LC (Fmab 4)

### Binding ELISAs: Procedure:

The fusion Mab was tested for its ability to bind to its targets in three different ELISAs: 1) EGFR1 target-binding ELISA, 2) TGFβ-target binding ELISA and 3) Bifunctional ELISA.

For the target binding ELISAs, the targets (rhEGFR-Fc chimera or TGFβ) were coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. Binding of the fusion Mab was detected by the addition of a biotinylated anti-human IgG F(ab)₂ secondary antibody, followed by a 1 hr incubation with peroxidase-conjugated streptavidin at room temperature. TMB substrate solution was added and the reaction stopped with IN H₂SO₄. The absorbance was measured at 450 nm on a BioTek Synergy H4 Hybrid reader.

For the bifunctional ELISA, rhEGFR-Fc chimera was coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. After washing, TGFβ was added and the plate was incubated at room temperature for 1 hr. The plate was washed and anti-TGFβ-biotin was added and the plate incubated at room temperature for 1 hr. The plate was washed and streptavidin-HRP was added and the plate incubated at room temperature for 1 hr. After washing, TMB substrate solution was added and the reaction stopped with IN H₂SO₄. The absorbance was measured at 450 nm on a BioTek Synergy H4 hybrid reader.

### Results:

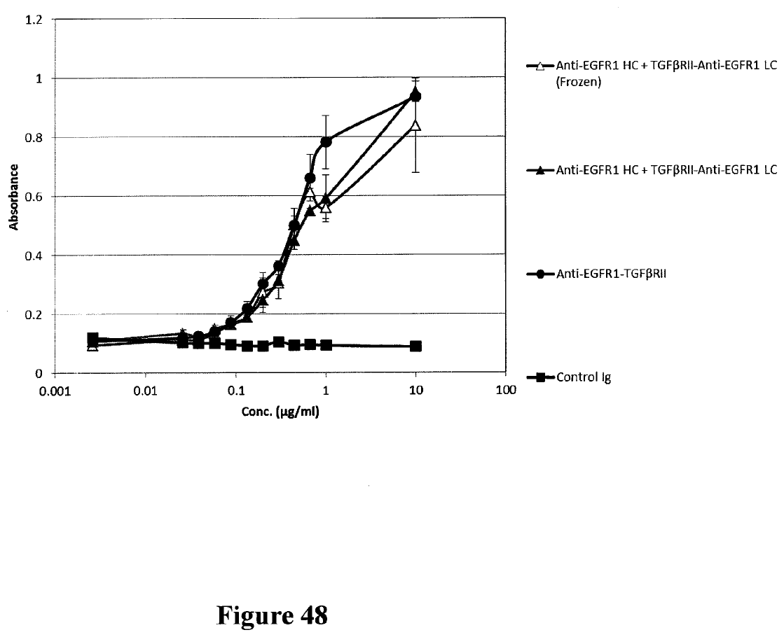

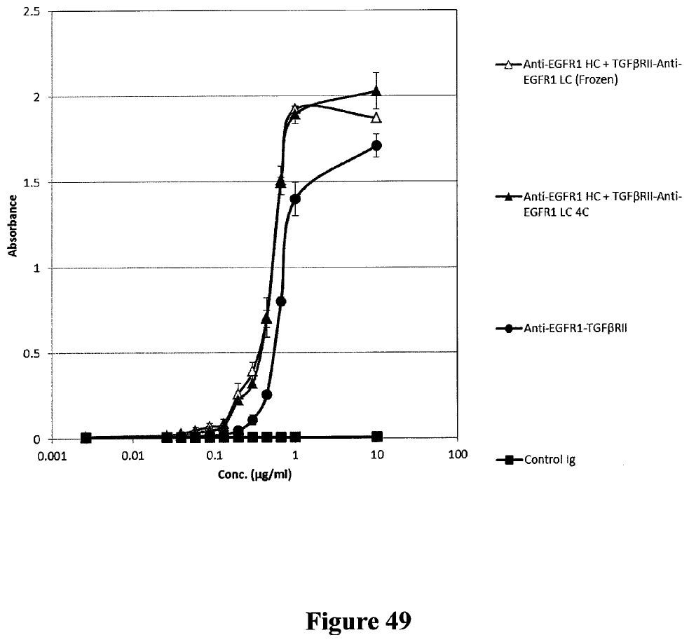

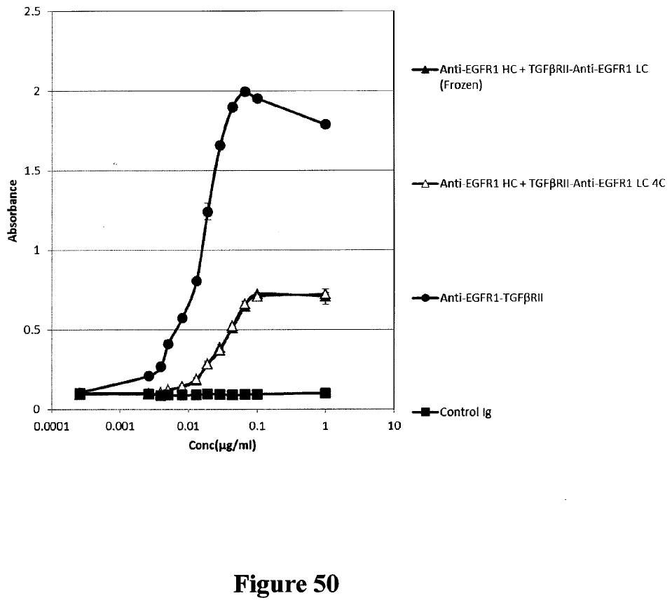

The binding of the Anti-EGFR1 HC+TGFβRII-Anti-EGFR1 LC fusion Mab to both the targets EGFR1 (FIG. 48) and TGFβ (FIG. 49) was comparable with anti-EGFR1-TGFβRII. The Anti-EGFR1 HC+TGFβRII-Anti-EGFR1 LC fusion Mab was also tested in a bifunctional ELISA to determine whether the anti-EGFR1 and TGFβRII domains of the Mab can bind to their respective targets without interfering with each other. As seen in FIG. 50, the binding of Anti-EGFR1 HC+TGFβRII-Anti-EGFR1 LC fusion Mab is reduced as compared to anti-EGFR1-TGFβRII, suggesting that there is some interference in binding to either target due to the construction of the fusion Mab.

### Binding to Cells Expressing EGFR1

### Procedure:

A-431 cells were grown in flasks until they reached 70-80% confluency. The cells were trypsinized and harvested. The cells were stained with different dilutions of Anti-EGFR1 HC+TGFβRII-Anti-EGFR1 LC fusion Mab or control Ig at 2-8° C. for 30 minutes. The cells were washed and incubated with anti-Human IgG-FITC conjugate at 2-8° C. for 30 minutes. After washing, the cells were analyzed on a flow cytometer. Live cells were gated based on their FSC vs SSC profiles. The total MFI for the gated population were recorded.

### Results:

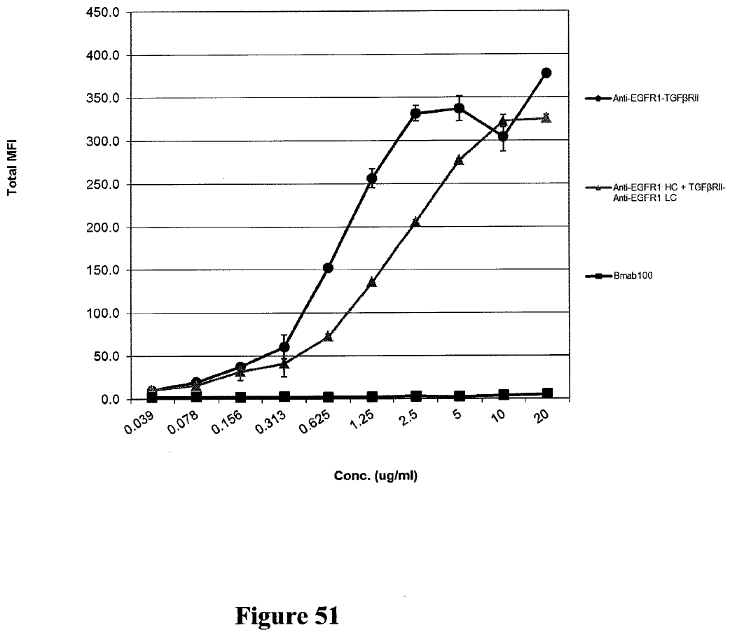

The Anti-EGFR1 HC+TGFβRII-Anti-EGFR1 LC fusion binds to EGFR-expressing A-431 cells in a dose dependent manner (FIG. 51). The binding of Anti-EGFR1 HC+TGFβRII-Anti-EGFR1 LC is reduced compared to the binding of anti-EGFR1-TGFβRII.

### 8. Anti-EGFR1 HC-TGFβRII+TGFβRII-Anti-EGFR1 LC (Fmab 10)

### Binding ELISAs: Procedure:

The fusion Mab was tested for its ability to bind to its targets in three different ELISAs: 1) EGFR1 target-binding ELISA, 2) TGFβ-target binding ELISA and 3) Bifunctional ELISA.

For the target binding ELISAs, the targets (rhEGFR-Fc chimera or TGFβ) were coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. Binding of the fusion Mab was detected by the addition of a biotinylated anti-human IgG F(ab)₂ secondary antibody, followed by a 1 hr incubation with peroxidase-conjugated streptavidin at room temperature. TMB substrate solution was added and the reaction stopped with IN H₂SO₄. The absorbance was measured at 450 nm on a BioTek Synergy H4 Hybrid reader.

For the bifunctional ELISA, rhEGFR-Fc chimera was coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. After washing, TGFβ was added and the plate was incubated at room temperature for 1 hr. The plate was washed and anti-TGFβ-biotin was added and the plate incubated at room temperature for 1 hr. The plate was washed and streptavidin-HRP was added and the plate incubated at room temperature for 1 hr. After washing, TMB substrate solution was added and the reaction stopped with 1N H₂SO₄. The absorbance was measured at 450 nm on a BioTek Synergy H4 hybrid reader.

### Results:

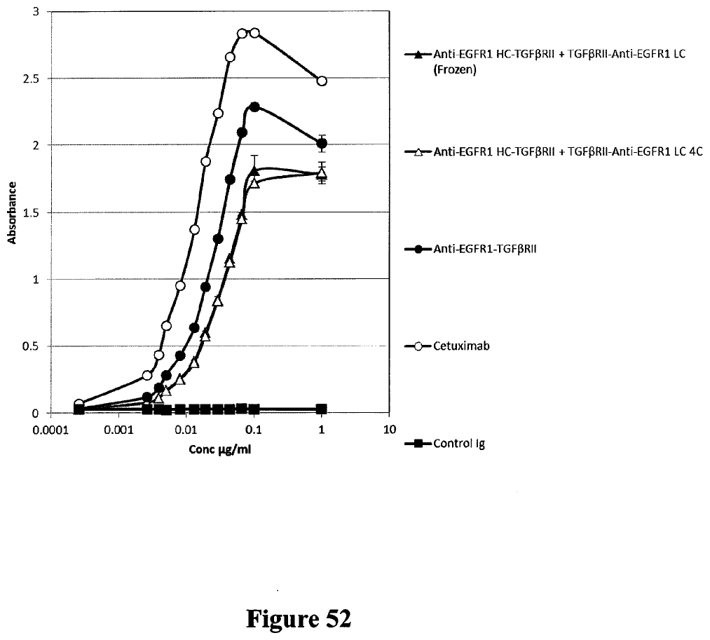

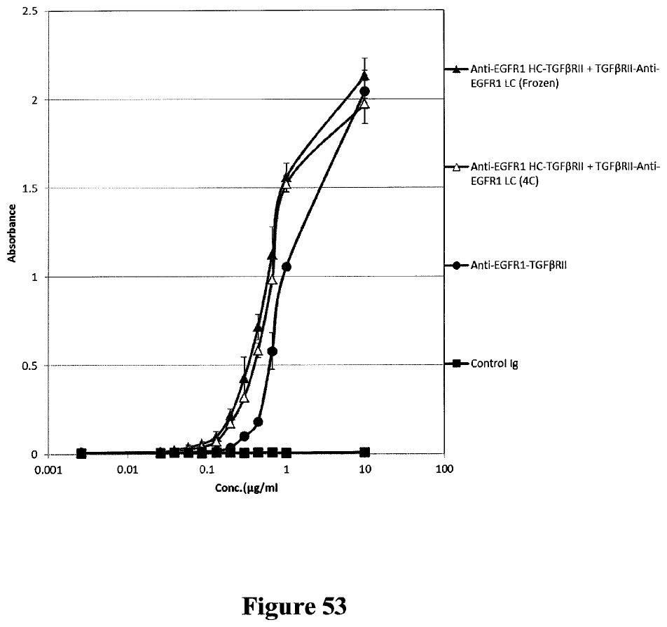

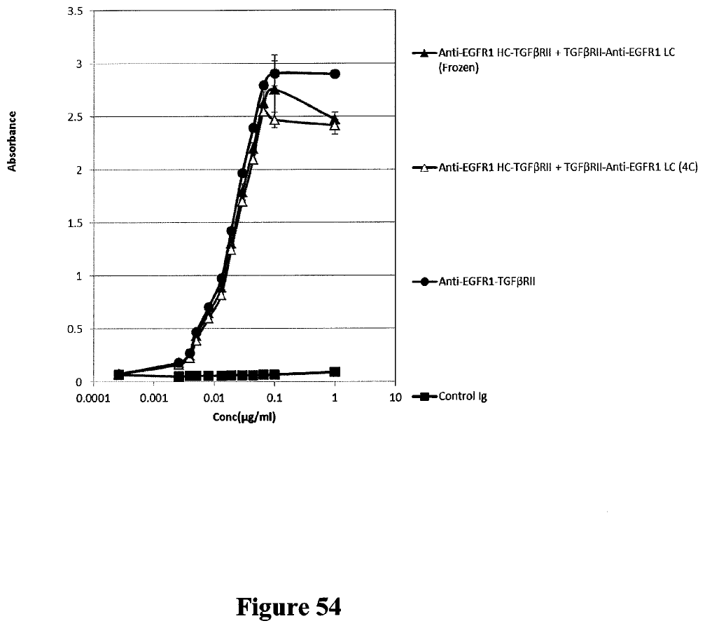

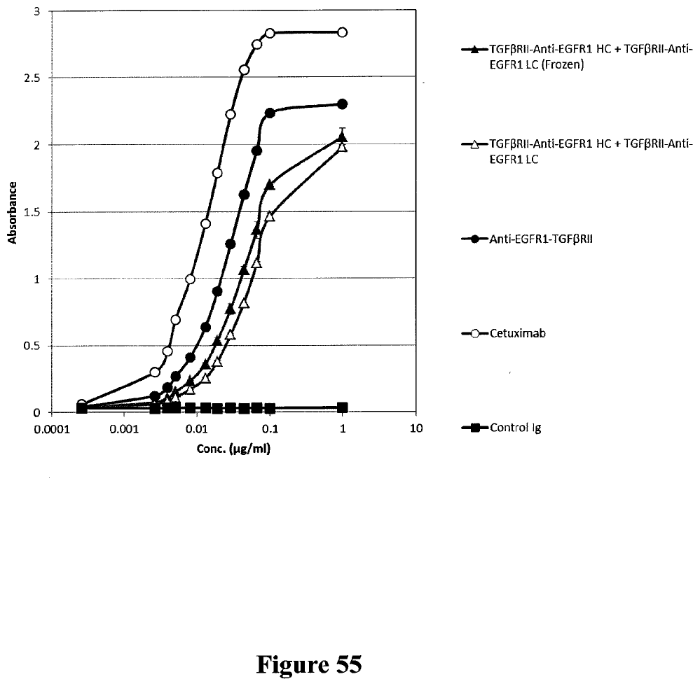

The binding of Anti-EGFR1 HC-TGFβRII+TGFβRII-Anti-EGFR1 LC fusion Mab to the target EGFR1 was slightly reduced (FIG. 52) but was higher for TGFβ (FIG. 53) when compared to the binding of anti-EGFR1-TGFβRII. The Anti-EGFR1-TGFβRII+TGFβRII-Anti-EGFR1 LC fusion Mab was also tested in a bifunctional ELISA to determine whether the anti-EGFR1 and TGFβRII domains of the Mab can bind to their respective targets without interfering with each other. As seen in FIG. 54, the binding of Anti-EGFR1 HC-TGFβRII+TGFβRII-Anti-EGFR1 LC fusion Mab is comparable to anti-EGFR1-TGFβRII, suggesting that there is no interference in binding to either target due to the construction of the fusion Mab.

### 9. TGFβRII-Anti-EGFR1 HC+TGFβRII-Anti-EGFR1 LC (Fmab 12)

### Binding ELISAs: Procedure:

The fusion Mab was tested for its ability to bind to its targets in three different ELISAs: 1) EGFR1 target-binding ELISA, 2) TGFβ-target binding ELISA and 3) Bifunctional ELISA.

For the target binding ELISAs, the targets (rhEGFR-Fc chimera or TGFβ) were coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. Binding of the fusion Mab was detected by the addition of a biotinylated anti-human IgG F(ab)₂ secondary antibody, followed by a 1 hr incubation with peroxidase-conjugated streptavidin at room temperature. TMB substrate solution was added and the reaction stopped with IN H₂SO₄. The absorbance was measured at 450 nm on a BioTek Synergy H4 Hybrid reader.

For the bifunctional ELISA, rhEGFR-Fc chimera was coated onto NUNC maxisorb plates overnight at 4° C. The plates were washed and then blocked with superblock at room temperature for 2 hr. Different dilutions of the fusion Mab or the negative control antibody was added to the plate. The plate was incubated at room temperature for 1 hr. After washing, TGFβ was added and the plate was incubated at room temperature for 1 hr. The plate was washed and anti-TGFβ-biotin was added and the plate incubated at room temperature for 1 hr. The plate was washed and streptavidin-HRP was added and the plate incubated at room temperature for 1 hr. After washing, TMB substrate solution was added and the reaction stopped with 1N H₂SO₄. The absorbance was measured at 450 nm on a BioTek Synergy H4 hybrid reader.

### Results:

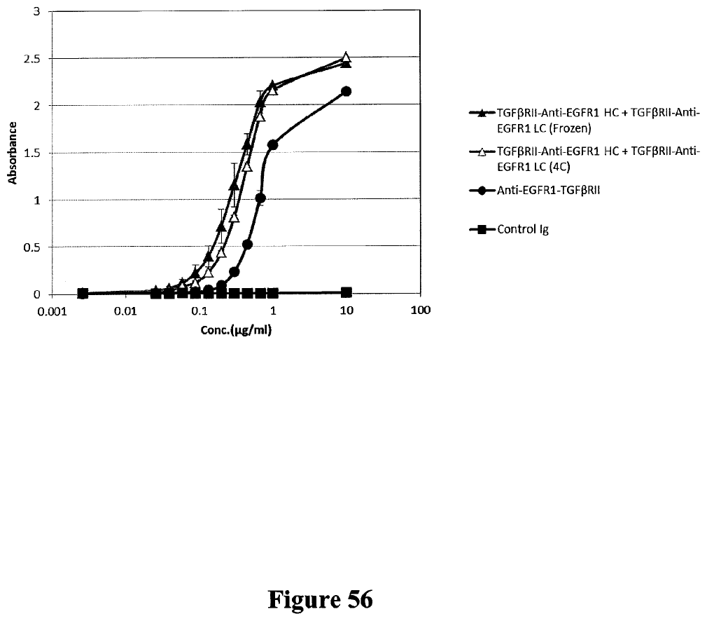

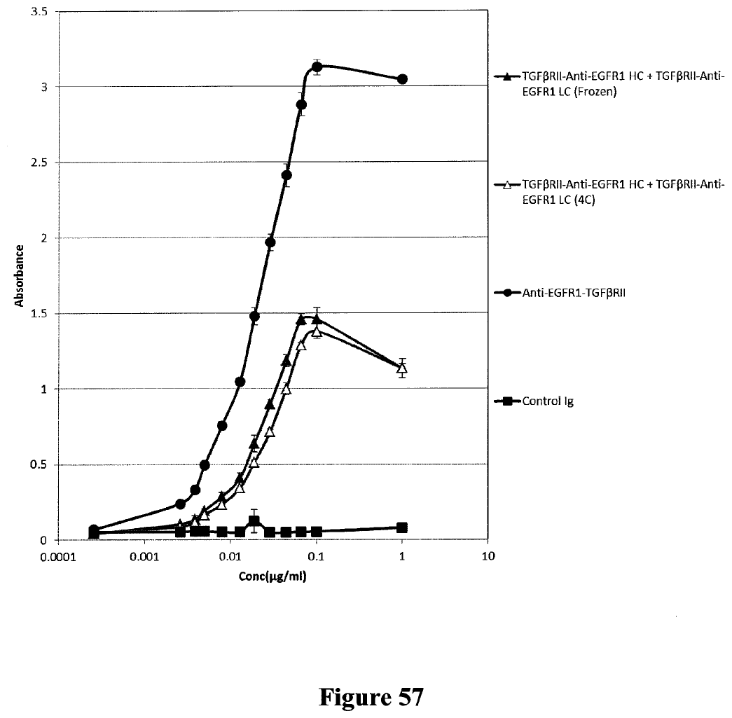

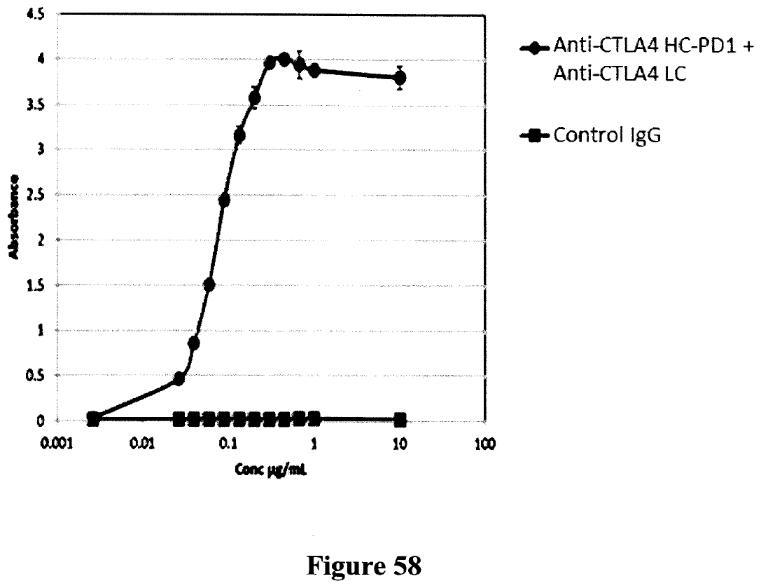

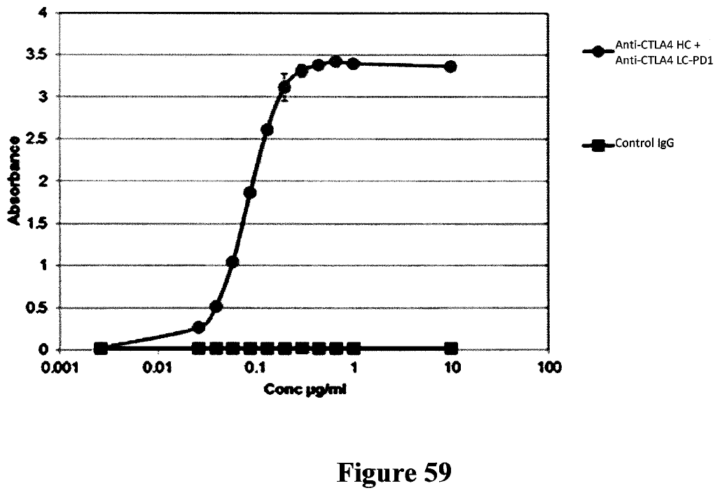

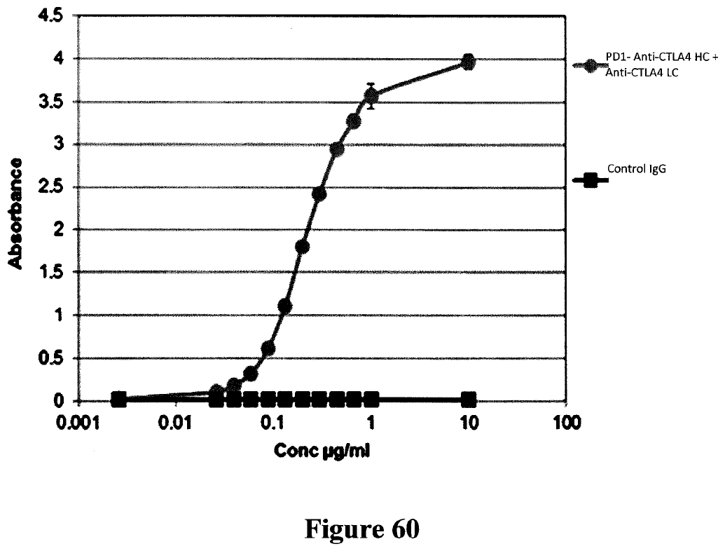

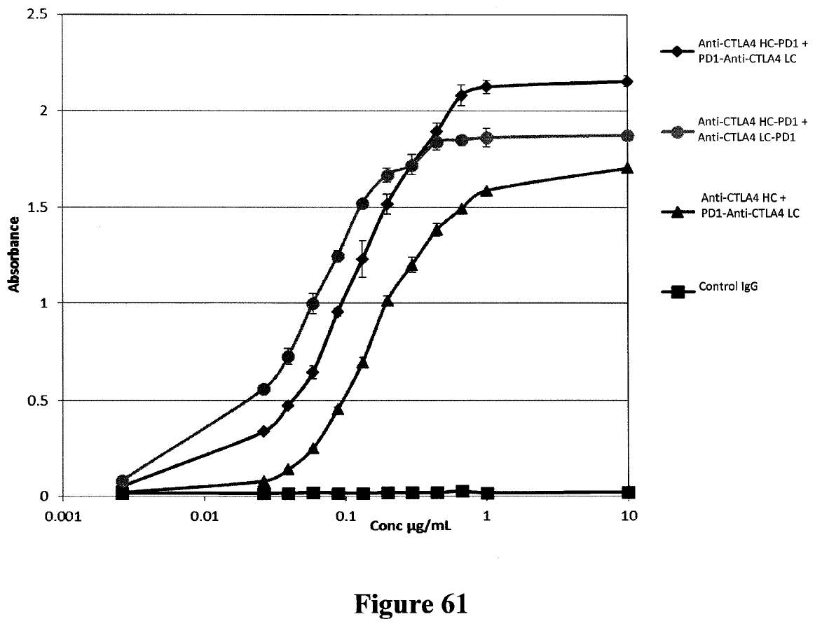

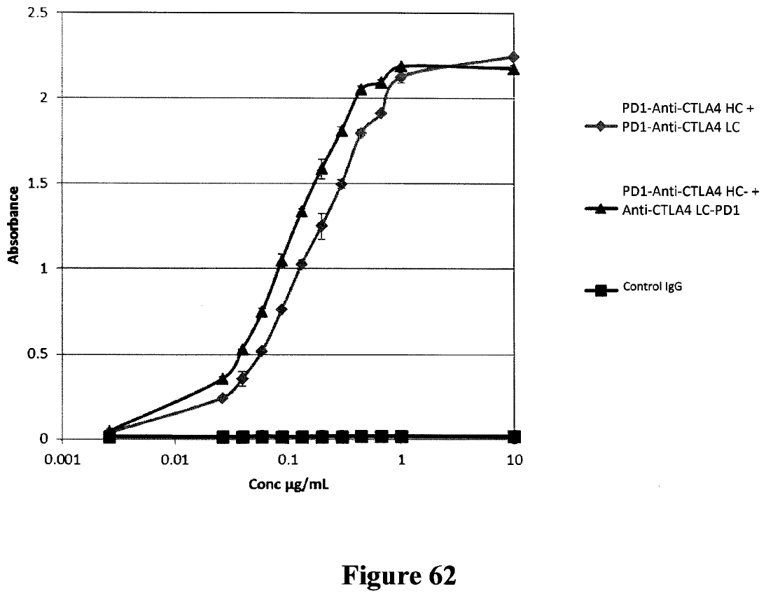

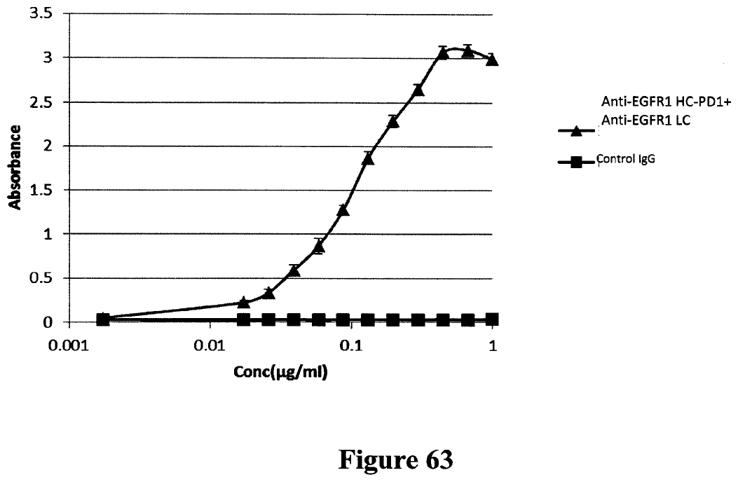

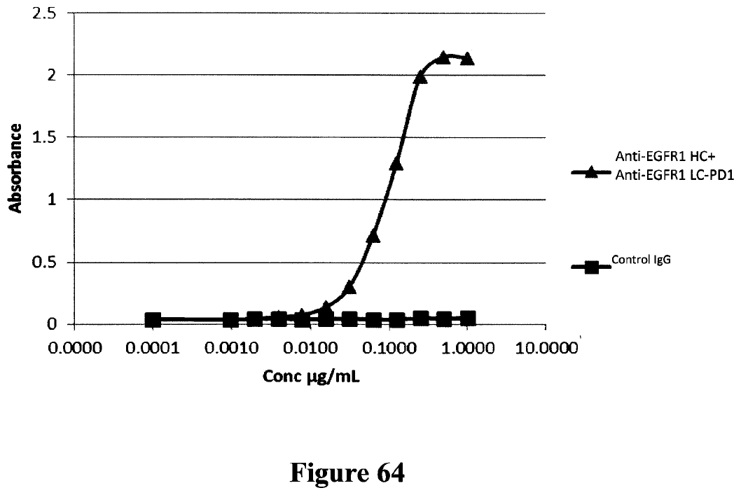

The binding of TGFβRII-Anti-EGFR1 HC+TGFβRII-Anti-EGFR1 LC fusion Mab to the target EGFR1 was slightly reduced (FIG. 53) but was higher for TGFβ (FIG. 56) when compared to the binding of anti-EGFR1-TGFβRII. The TGFβRII-Anti-EGFR1 HC+TGFβRII-Anti-EGFR1 LC fusion Mab was also tested in a bifunctional ELISA to determine whether the anti-EGFR1 and TGFβRII domains of the Mab can bind to their respective targets without interfering with each other. As seen in FIG. 57, the binding of TGFβRII-Anti-EGFR1 HC+TGFβRII-Anti-EGFR1 LC fusion Mab is reduced compared to anti-EGFR1-TGFβRII, suggesting that there is interference in binding to either target due to the construction of the fusion Mab.

### REFERENCES

The contents of all references cited herein are incorporated by reference herein for all purposes.
Altschul, et al., 1990, Basic local alignment search tool, J. Mol. Biol. 15:403-10.
Ausubel, et al., (ed), 1998, Current Protocols in Molecular Biology, Vol. 2, Ch. 13, Greene Publish. Assoc. & Wiley Interscience.
Bitter, et al., 1987, Expression and Secretion Vectors for Yeast; Methods in Enzymology, 153:5 16-544.
Bitter, 1987, Heterologous Gene Expression in Yeast, Methods in Enzymology, Vol. 152, pp. 673-684.
Dong M., et al., 2006, Role of transforming growth factor-β in hematologic malignancies. Blood. 107:4589-4596.
Deutscher, M., et al., Guide to Protein Purification: Methods in Enzymology, Vol. 182, Academic Press (1990).
Flies, et al., 2011, Blockade of the B7-H1/PD-1 Pathway for Cancer Immunotherapy, J. Biol Med., 84 (4): 409-421.
Glover, 1986, DNA Cloning, Vol. II, Ch. 3, IRL Press, Wash., D.C.
Gluzman (ed.), 1982, Eukaryotic Viral Vectors, Cold Spring Harbor, N.Y.: Cold Spring Harbor Laboratory.
Grant, et al., 1987, Expression and Secretion Vectors for Yeast, Methods in Enzymology, Vol. 153, 516-544.
Lee, et al., 1994, Delivery of liposomes into cultured KB cells via folate receptor-mediated endocytosis. J. Biol. Chem., 269:3 198.
Lee, et al., 1995, Folate-mediated tumor cell targeting of liposome-entrapped doxorubicin in vitro. Biochem. Biophys. Actu, 1233: 134-144.
Mackett, et al., 1982, Vaccinia virus: a selectable eukaryotic cloning and expression vector, Proc. Natl. Acad. Sci. USA, 79:7415-7419.
Mackett, et al., 1984, General method for production and selection of infectious vaccinia virus, J. Virol., 49:857-864.
Nomi, et al., 2007, Clinical significance and therapeutic potential of the programmed death-1 ligand/programmed death-1 pathway in human pancreatic cancer. Clin Cancer Res., 13:2151-7.
Okazaki, et al., 2007, PD-1 and PD-1 ligands: from discovery to clinical Application. International Immunology, Vol. 19, No. 7, pp. 813-824.
Panicali, et al., 1982, Construction of poxviruses as cloning vectors: insertion of the thymidine, Proc. Natl. Acad. Sci. USA, 79:4927-4931.
Pardoll, D M., 2012, The blockage of immune checkpoints in cancer immunotherapy. Nat. Rev. Cancer, 12(4): 252-64.
Strathem, et al., 1982, The Molecular Biology of the Yeast Saccharomyces, Cold Spring Harbor Press, Vols. I and II.
Thompson R H, et al., 2004, Costimulatory B7-H1 in renal cell carcinoma patients: Indicator of tumor aggressiveness and potential therapeutic target. Proc Natl Acad Sci USA., 101 (49): 17174-9.
Teicher B A., 2001, Malignant cells, directors of the malignant process: role of transforming growth factor beta. Cancer Metastasis Rev, 20:133-143.
Wrzesinski, et al., 2007, Transforming Growth Factor-β and the Immune Response: Implications for Anticancer Therapy, Clin Cancer Res, 13; 5262.

1-2. (canceled)

3. A method for preparing a therapeutically active fusion protein, wherein the fusion protein comprises a tumor targeting moiety and an immunomodulatory molecule, wherein the immunomodulatory molecule counteracts immune tolerance of a cancer cell,
wherein the tumor targeting moiety is an antibody that binds to epidermal growth factor 1 (EGFR1) and wherein the fusion protein is prepared by the following steps:
(a) preparing a codon optimized nucleotide sequence encoding the fusion protein,
wherein the codon optimized nucleotide sequence has been modified to increase CG sequences,
wherein the codon optimized nucleotide sequence for the antibody is lacking nucleotides for expression of a lysine at the C-terminal end of the heavy chain of the antibody,
wherein the immunomodulatory molecule binds to Programmed Death Ligand-1 (PDL1) and
wherein the codon optimized nucleotide sequences consist of SEQ ID NOs: 1, 2, 4 and 7;
(b) cloning the codon optimized nucleotide sequence of said fusion protein in a CHO host cell capable of transient or stable expression;
(c) growing the host cell in a media under suitable conditions for growing and allowing the host cell to express the fusion protein; and
(d) collecting secreted fusion proteins and optionally for further purification.

4. The method of claim 3, wherein the immunomodulatory molecule is linked to the antibody by an amino acid sequence of sufficient length to allow bi-specific binding of the fusion protein, wherein the linker is encoded by SEQ ID NO: 4.

5. The method of claim 3, wherein the immunomodulatory molecule is linked directly or through a linker to the heavy chain of the antibody, light chain of the antibody or both chains.

6. The method of claim 5, wherein the immunomodulatory molecule is linked through a linker to the heavy chain of the antibody, light chain of the antibody or both chains, wherein the linker is encoded by SEQ ID NO: 4.

7. The method of claim 3, wherein the immunomodulatory molecule is linked directly or through a linker to the N or C terminus of the heavy chain of the antibody, N or C terminus of the light chain of the antibody or both N and C terminus of both chains.

8. The method of claim 7, wherein the immunomodulatory molecule is linked through a linker to the N or C terminus of the heavy chain of the antibody, N or C terminus of the light chain of the antibody or both N and C terminus of both chains, wherein the linker is encoded by SEQ ID NO: 4.

9. A method of treating cancer in a subject, the method comprising:
(a) preparing a therapeutically active fusion protein having anti-proliferative activity, wherein the fusion protein comprises a tumor targeting moiety and at least one immunomodulatory molecule, wherein the tumor targeting moiety is an antibody that binds to EGFR1 and wherein the fusion protein is prepared by the following steps:
(i) preparing a codon optimized nucleotide sequence encoding the fusion protein, wherein the codon optimized nucleotide sequence has been modified to increase CG sequences, wherein the codon optimized nucleotide sequence for the antibody is lacking nucleotides for expression of a lysine at the C-terminal end of the heavy chain of the antibody, wherein the immunomodulatory molecule binds to PD-L1 and wherein the codon optimized nucleotide sequences consisting of SEQ ID NOs: 1, 2, 4 and 7;
(ii) cloning the codon optimized nucleotide sequence of said fusion protein in a CHO host cell capable of transient or stable expression;
(iii) growing the host cell in a media under suitable conditions for growing and allowing the host cell to express the fusion protein; and
iv) collecting secreted fusion proteins for optional purification; and
b) administering a therapeutically active amount of the fusion proteins to the subject.

10. The method of claim 9, wherein the immunomodulatory molecule is linked to the antibody by an amino acid sequence of sufficient length to allow bi-specific binding of the fusion protein, wherein the linker is encoded by SEQ ID NO: 4.
